(19) United States
(12) Patent Application Publication
(10) Pub. No.: US 2025/0090325 A1
(43) Pub. Date: Mar. 20, 2025

(54) APPARATUSES FOR AT LEAST PARTIALLY REPAIRING A REGURGITANT HEART VALVE

(71) Applicant: THE CLEVELAND CLINIC FOUNDATION, Cleveland, OH (US)

(72) Inventors: Jose Navia, Cleveland, OH (US); Samir Kapadia, Cleveland, OH (US); Torey Hovest, Cleveland, OH (US)

(21) Appl. No.: 18/729,534

(22) PCT Filed: Jan. 18, 2023
(86) PCT No.: PCT/US2023/011010

§ 371 Date: Jul. 17, 2024

Related U.S. Application Data
(60) Provisional application No. 63/300,300, filed on Jan. 18, 2022.
(60) Provisional application No. 63/311,408, filed on Feb. 17, 2022.

(51) Int. Cl. A61F 2/24

(52) U.S. Cl. CPC A61F 2/2463; A61F 2210/0014; A61F 2210/0057; A61F 2220/0016; A61F 2220/0025

(57) ABSTRACT

An apparatus for at least partially repairing a regurgitant heart valve includes at least one subvalvular device defining a longitudinal axis and including a subvalvular portion located adjacent a subvalvular cardiac wall adjacent to the heart valve. An anchor portion is adjacent to, and is longitudinally spaced from, the subvalvular portion. A connector neck is interposed longitudinally between, and is attached to both of, the subvalvular portion and the anchor portion. The connector neck penetrates longitudinally through at least one of a base of the first valve leaflet and an annulus of the heart valve at a manufactured puncture site. A fastener is selectively connected to the at least one subvalvular device. The fastener penetrates through at least one of the first and second valve leaflets to maintain the at least one leaflet in a predetermined position with respect to the at least one subvalvular device.

### RELATED APPLICATIONS

This application claims priority from U.S. Provisional Application No. 63/300,300, filed 18 Jan. 2022, and from U.S. Provisional Application No. 63/311,408, filed 17 Feb. 2022, the subject matter of both of which is incorporated herein by reference in its entirety.

### TECHNICAL FIELD

This disclosure relates to apparatuses and methods for repairing a heart valve and, more particularly, to methods and apparatuses for providing a subvalvular device to at least partially repair a regurgitant heart valve.

### BACKGROUND

Functional Mitral Regurgitation (FMR) and Secondary Tricuspid Regurgitation (STR) are conditions resulting from anatomic dilatation in the shape of the heart caused by ischemia, infarctions, left-sided heart disease, or pulmonary hypertension. FMR and STR are not diseases affecting the cardiac valve leaflets or the valves themselves, but rather involve a ventricle dilation, deformation, and/or displacement which causes the chordae to tether the leaflet and misplaces the normal leaflet coaptation, therefore allowing for bloodflow back into the left or right atrium (i.e., regurgitation). Despite the distinct cause of the regurgitations, many medical interventions still revolve around replacement or repairing the valve with a prosthetic valve or leaflet manipulation, including resection, folding and suturing of the leaflets, clips to pull together the leaflet tissues, and artificial chordae. However, none of these methods have had a desired subvalvular therapeutic effect to date.

### SUMMARY

In an aspect, alone or in combination with any other aspect, an apparatus for at least partially repairing a regurgitant heart valve is described. The heart valve includes at least first and second valve leaflets. The apparatus comprises at least one subvalvular device defining a longitudinal axis and including a subvalvular portion located adjacent a subvalvular cardiac wall adjacent to the heart valve. An anchor portion is adjacent to, and is longitudinally spaced from, the subvalvular portion. A connector neck is interposed longitudinally between, and is attached to both of, the subvalvular portion and the anchor portion. The connector neck penetrates longitudinally through at least one of a base of the first valve leaflet and an annulus of the heart valve at a manufactured puncture site. A fastener is selectively connected to the at least one subvalvular device. The fastener penetrates through at least one of the first and second valve leaflets to maintain the at least one leaflet in a predetermined position with respect to the at least one subvalvular device.

### BRIEF DESCRIPTION OF THE DRAWINGS

For a better understanding, reference may be made to the accompanying drawings, in which:

FIG. 1 is a schematic partial side view of an apparatus according to an aspect of the present invention;

FIGS. 2-4 schematically depict an example sequence of operation of the apparatus of FIG. 1;

FIG. 5 is a schematic detail view of the apparatus of FIG. 1;

FIG. 6 is a schematic partial side view of the apparatus of FIG. 1, in a first alternate configuration;

FIG. 7 is a schematic side view of the apparatus of FIG. 1, in a second alternate configuration;

FIG. 8 is a schematic detail view of the apparatus of FIG. 7;

FIG. 9 is a schematic detail view of the apparatus of FIG. 7;

FIGS. 10-12 schematically depict an example sequence of operation of the apparatus of FIG. 1;

FIG. 13 is a schematic side view of the apparatus of FIG. 1, in a third alternate configuration;

FIGS. 14-15 schematically depict an example sequence of operation of the apparatus of FIG. 7;

FIGS. 16-18 schematically depict example installation configurations of the apparatus of FIG. 7;

FIG. 19 is a schematic side view of an example installation tool for the apparatus of FIG. 1;

FIGS. 20-21 are schematic side views of example arrangements of the apparatus of FIG. 1;

FIG. 22 is a side view of a component of the apparatus of FIG. 1, in a first configuration;

FIG. 23 is a perspective front view of the component of FIG. 22 in an expanded condition;

FIG. 24 is a perspective front view of the component of FIG. 22 in a collapsed condition;

FIGS. 25-26 schematically partially depict a deployment of the component of FIG. 22;

FIG. 27 is a schematic side view of the component of FIG. 22 in an installed condition;

FIG. 28 is a perspective front view of a component of the apparatus of FIG. 1 in a second configuration and a collapsed condition;

FIG. 29 schematically depicts the component of FIG. 28 in an expanded condition during installation thereof;

FIG. 30 is a schematic front perspective view of a component of the apparatus of FIG. 1 in a third configuration;

FIG. 31 schematically depicts the component of FIG. 30 in a collapsed condition during installation thereof;

FIGS. 32 and 34-35 schematically depict an example sequence of operation of a component of the apparatus of FIG. 1 in a fourth configuration;

FIG. 33 is a cross-sectional view taken along line 33-33 of FIG. 32;

FIG. 34 is a schematic side view of the component of FIG. 32 in an expanded condition;

FIG. 36 is a schematic front view of a component of the apparatus of FIG. 1 in a fifth configuration;

FIG. 37 is a schematic side view of the component of FIG. 36 in a partially expanded condition during installation thereof;

FIG. 38 is a schematic side view of a component of the apparatus of FIG. 1 in a sixth configuration;

FIG. 39 is a schematic side view of a component of the apparatus of FIG. 1 in a seventh configuration;

FIG. 40 is a schematic side view of a component of the apparatus of FIG. 1 in a eighth configuration;

FIG. 41 is a schematic top perspective view of an apparatus according to an aspect of the present invention, in an example use environment;

FIG. 42 is a schematic top perspective view of the apparatus of FIG. 41 in a second configuration;

FIG. 43 is a schematic side view of the apparatus of FIG. 1, in a fourth alternate configuration;

FIG. 44 is a schematic top view of the apparatus of FIG. 43 in an example use environment;

FIG. 45 is a schematic side view of the apparatus of FIG. 1, in a fifth alternate configuration and in a first example use environment;

FIG. 46 is a schematic side view of the apparatus of FIG. 45, in a second example use environment; and

FIG. 47 is a schematic side view of the apparatus of FIG. 45, in a third example use environment.

### DESCRIPTION OF ASPECTS OF THE DISCLOSURE

Unless defined otherwise, all technical and scientific terms used herein have the same meaning as is commonly understood by one of ordinary skill in the art to which the present disclosure pertains.

As used herein, the term “subject” can be used interchangeably with the term “patient” and refer to any warm-blooded organism including, but not limited to, human beings, pigs, rats, mice, dogs, goats, sheep, horses, monkeys, apes, rabbits, cattle, farm animals, livestock, etc.

As used herein, the terms “treat” or “treating” can refer to therapeutically regulating, preventing, improving, alleviating the symptoms of and/or reducing the effects of a leaflet coaptation issue. As such, treatment also includes situations where a leaflet coaptation issue, or at least a symptom associated therewith, is completely inhibited, e.g., prevented from happening or stopped (e.g., terminated) such that the subject no longer suffers from the leaflet coaptation issue, or at least the symptom(s) associated therewith.

As used herein, the singular forms “a”, “an”, and “the” can include the plural forms as well, unless the context clearly indicates otherwise. It will be further understood that the terms “comprises” and/or “comprising”, as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on”, “attached” to, “connected” to, “coupled” with, “contacting”, “adjacent”, etc., another element, it can be directly on, attached to, connected to, coupled with, contacting, or adjacent the other element, or intervening elements may also be present. In contrast, when an element is referred to as being, for example, “directly on”, “directly attached” to, “directly connected” to, “directly coupled” with, “directly contacting”, or “directly adjacent” another element, there are no intervening elements present. It will also be appreciated by those of ordinary skill in the art that references to a structure or feature that is disposed “directly adjacent” another feature may have portions that overlap or underlie the adjacent feature, whereas a structure or feature that is disposed “adjacent” another feature might not have portions that overlap or underlie the adjacent feature.

Spatially relative terms, such as “under”, “below”, “lower”, “over”, “upper”, “proximal”, “distal”, and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

As used herein, the phrase “at least one of X and Y” can be interpreted to include X, Y, or a combination of X and Y. For example, if an element is described as having at least one of X and Y, the element may, at a particular time, include X, Y, or a combination of X and Y, the selection of which could vary from time to time. In contrast, the phrase “at least one of X” can be interpreted to include one or more Xs.

It will be understood that, although the terms “first”, “second”, etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from about 1 to about 6 should be considered to have specifically disclosed subranges such as from 1 to 3, from 1 to 4, from 1 to 5, from 2 to 4, from 2to 6, from 3 to 6 etc., as well as individual and partial numbers within that range, for example, 1, 1.1, 2, 2.8, 3, 3.2, 4, 4.7, 4.9, 5, 5.5 and 6. This applies regardless of the breadth of the range.

The invention comprises, consists of, or consists essentially of the following features, in any combination.

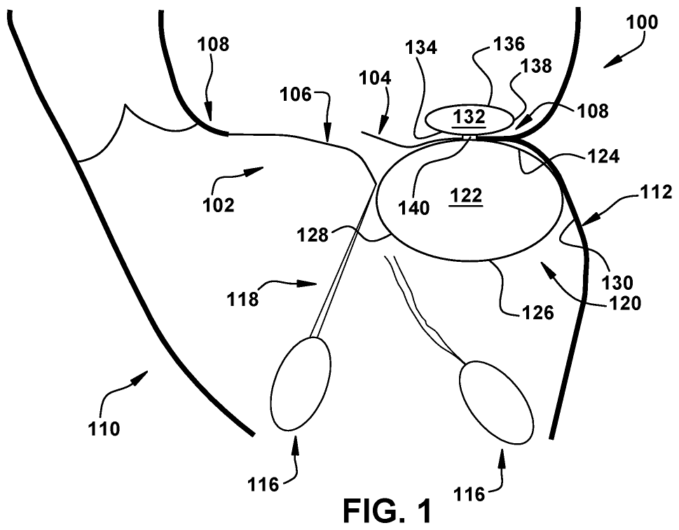

FIG. 1 depicts at least a portion of an apparatus 100 for at least partially repairing a regurgitant heart valve 102. The heart valve 102 includes at least first and second valve leaflets 104 and 106, respectively, carried in a valve annulus 108. The heart valve 102 is located in a heart 110 having an outer wall 112 including a myocardium 114. The heart valve 102, first and second valve leaflets 104 and 106, valve annulus 108, and myocardium 114 all comprise “cardiac tissue” (along with various other patient tissues, as are known to one of ordinary skill in the art) and will be referenced collectively thereas. For example, the cardiac tissue can also include papillary muscles 116 and chordae tendineae 118. While a two-leaflet valve (e.g., a mitral valve) 102 is referenced in the below description and shown in many of the Figures for ease of depiction, it is also contemplated that the apparatus 100 may be used in a three-leaflet valve (e.g., a tricuspid valve) or any other desired patient tissue use environment, whether or not associated with cardiac tissue. One of ordinary skill in the art will readily be able to configure the apparatus 100 and associated structures for any desired use environment.

The apparatus 100 may be used for at least partially repairing a regurgitant heart valve 102. The apparatus 100 includes at least one subvalvular device 120 defining a longitudinal axis A. The subvalvular device 120 may be substantially similar to any of those shown and described in any of U.S. Pat. No. 10,537,432, issued 21 Jan. 2020; U.S. Pat. No. 11,241,313, issued 8 Feb. 2022; and/or copending U.S. patent application Ser. No. 17/396,987, filed 9 Aug. 2021 (collectively referenced hereafter as “the '432 family”); all of which are titled “Apparatuses and Methods for At Least Partially Supporting a Valve Leaflet of a Regurgitant Heart Valve” and all of which are incorporated herein by reference in their entirety for all purposes.

The subvalvular device 120 includes a subvalvular portion 122 including an upper subvalvular surface 124 longitudinally spaced from an oppositely facing lower subvalvular surface 126. The upper subvalvular surface 124 is configured for placement selectively adjacent, for example, in contact with, an underside of a first valve leaflet 104, when the subvalvular device 102 is installed at the heart valve 102. A subvalvular perimeter wall 128 extends longitudinally between, and is integrally and contiguously formed with both of, the upper and lower subvalvular surfaces 124 and 126. At least a portion of the subvalvular portion 122, such as the subvalvular perimeter wall 128, is located adjacent, optionally in contact with, a subvalvular cardiac wall 130 which is adjacent to the heart valve 102 (e.g., located beneath the heart valve) when the subvalvular device 120 is installed at the heart valve 102.

Particularly when the subvalvular perimeter wall 128 is in contact with the subvalvular cardiac wall 130, the subvalvular portion 122 may act to “bolster” or “prop up” the first valve leaflet 104 from below, braced against the subvalvular cardiac wall 130, during at least a portion of the cardiac cycle. This support from the subvalvular portion 122, when present, will tend to reduce regurgitation of the heart valve 102 by pushing the first valve leaflet 104 into apposition with the second valve leaflet 106. It is contemplated, however, that the subvalvular device 120, or structures thereof, could instead be configured (e.g., via size, hoop strength, location, and/or any other factors) to provide the below-described anchoring portions with any potential effect on positioning of the first valve leaflet 104 being incidental and/or limited to a predetermined portion of the cardiac cycle. Though “first” and “second” valve leaflets are referenced herein, for ease of discussion, it should be understood that these are arbitrary designations and that any leaflet of a heart valve 102 of any type could be associated with any portion of the apparatus 100 according to the description herein.

An anchor portion 132 is adjacent to, and longitudinally spaced from, the upper subvalvular surface 124. The anchor portion 132 includes a lower anchor surface 134 longitudinally spaced from an oppositely facing upper anchor surface 136. The lower anchor surface 134 is configured for selectively placement adjacent, for example, contacting, an upper side of a first valve leaflet 104, when the subvalvular device 102 is installed at the heart valve 102. An anchor perimeter wall 138 extends longitudinally between the upper and lower anchor surfaces 136 and 134.

It should be noted that the upper supporter and anchor surfaces 124 and 136, the lower supporter and anchor surfaces 126 and 134, and the subvalvular and anchor perimeter walls 128 and 138 are described herein as being different surfaces from one another. However, one of ordinary skill in the art will understand that either of the subvalvular portion 122 and the anchor portion 132 could be configured such that these surfaces and walls could not be clearly delineated from one another and that adjacent structures could “flow” or be contoured in such a way that a definitive border therebetween is not unequivocally defined. One of ordinary skill in the art will be able to readily determine whether or not a particular structure is performing the function of a named element, in practice, and then label that structure accordingly.

The subvalvular device 120 also includes a connector neck 140 interposed longitudinally between, and attached to both of, the upper subvalvular surface 124 and the lower anchor surface 134. The connector neck 140 penetrates longitudinally through at least one of a base of the first valve leaflet 104 and an annulus 108 of the heart valve 102 at a manufactured puncture site. A “manufactured puncture site” is used herein to indicate an area of cardiac tissue through which an aperture is naturally or artificially formed, before or during installation of the subvalvular device 120. A native valve commissure is not considered a “manufactured puncture site”, for the purposes of the below description. The disclosures of the '432 family provide additional example details regarding the subvalvular device 120 and its placement into the position shown in the Figures.

The subvalvular portion 122, the anchor portion 132, and/or the connector neck 140 may each be at least partially formed from at least one of braided mesh strands of a first configuration, braided mesh strands of a second configuration, a balloon, a plurality of longitudinally extending struts, a plurality of laterally extending struts, and/or any other material. It is contemplated that at least one of the subvalvular portion 122, the anchor portion 132, and the connector neck 140 may be made from a first material (e.g., a plurality of struts cut from a tubular base), and at least another one of the subvalvular portion 122, the anchor portion 132, and/or the connector neck 140 may be made from a second material (e.g., braided mesh strands of a first configuration). For reasons which will be explained below, it may be desirable to form at least one of the subvalvular portion 122 and the anchor portion 132 from a mesh or other material which is resistant to tearing or ripping after puncture, and also may lend some structural integrity sufficient to help resist a tensile force while maintaining an installed position without substantial deformation that would tend to occlude the heart valve 102.

The subvalvular portion 122, anchor portion 132, and connector neck 140 may collectively enclose a single contiguous interior volume (e.g., by being different-diameter portions of a single balloon, mesh, and/or tubular structure), or may be broken up into discrete structures and attached together to form the subvalvular device 120. For example, the subvalvular portion 122 and anchor portion 132 could each be at least one of a balloon and a mesh construct, and the connector neck 140 could be a hollow tube or solid rod attaching the subvalvular portion 122 and anchor portion 132 together. In most use environments, the connector neck 140 will be substantially smaller than either of the subvalvular portion 122 and anchor portion 132, such as by having an average cross-sectional area which is at least a minority, if not a superminority, of the size of an average cross-sectional area of at least one of the subvalvular portion 122 and anchor portion 132.

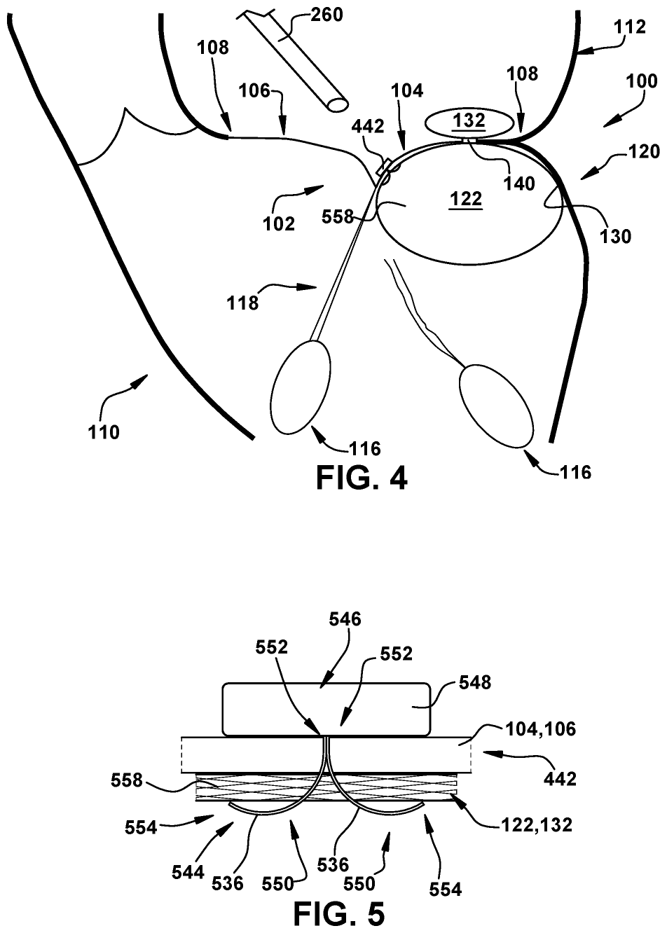

With reference now to FIG. 4, a fastener 442 may be selectively connected to the subvalvular device 120, such as being selectively connected to a selected one of the subvalvular portion 122 and the anchor portion 132. The fastener 442 penetrates into at least partially into a selected cardiac tissue to maintain at least one leaflet 104, 106 in a predetermined position with respect to at least another cardiac tissue and the subvalvular device 100. For example, the fastener 442 may penetrate through at least one of the first and second valve leaflets 104 and 106 to maintain at least one leaflet 104, 106 in a predetermined position with respect to at least a selected one of the subvalvular portion 122 and the anchor portion 132. This maintenance may occur via pinning/securement, application of tensile force, application of compressive force, and/or any other desired mechanism for a particular use environment. Numerous example aspects and configurations of various apparatuses 100 including example subvalvular devices 120 and fasteners 442 will be discussed below with reference to the Figures.

An example fastener 442 is shown in detail in FIG. 5. The fastener 442 includes at least one of a suture thread (i.e., a loop of elongated suture material), a pledget, a clip, a staple, a leg, a barb, a screw, any other desired fastener structure, or any combination thereof. As shown in FIG. 5, the fastener 442 may include a fastener anchor 544 for selective penetrative contact with any desired anchoring structure, such as, but not limited to, at least one cardiac tissue and/or the subvalvular portion 122, the anchor portion 132, or both of the at least one subvalvular device 120. The fastener 442 also includes a fastener head 546 for selective direct or indirect engagement with at least one cardiac tissue, such as at least one of the first and second valve leaflets 104 and 106.

As shown with the pledget-type fastener head 546 shown schematically in FIG. 5, fastener 442 may include a flexible covering 548 over at least a portion of an outer surface (e.g., as shown, the fastener head 546) of the fastener 442, for cushioning, promoting or discouraging tissue ingrowth, and/or any other desired purpose. For example, the flexible covering 548, when present, may include one or more of polyester, polytetrafluoroethylene (“PTFE”), expanded polytetrafluoroethylene (“ePTFE”), fabric, foam, allograft tissue, autograft tissue, cadaver tissue, an artificial or natural material membrane, any other flexible material, or any combination thereof. One of ordinary skill in the art can readily configure a fastener head 546 as desired for a particular use environment of the apparatus 100.

The fastener anchor 544 may include any desired structure which can be helpful in engaging at least one cardiac tissue and/or a portion of the subvalvular device 120. For example, the fastener anchor 544 can include one or more barbs, hooks, screws, adhesives, magnets, suture threads, staples, clips, pins, any other fastening means, or any combination thereof. In the example use environments shown in the Figures, the fastener anchor 544 penetrates at least partially into, and optionally completely through, at least one of a cardiac tissue and at least one portion of the subvalvular device 120. As shown in FIG. 5, this may be accomplished via the provision to the fastener anchor 544 of at least one elongate leg 550 having first and second leg ends 552 and 554, respectively, separated by a leg body 556. The first leg end 552 is attached (directly or indirectly) to the fastener head 546. The leg body 556 penetrates into, and optionally through, at least one of a cardiac tissue and a portion of the subvalvular device 120 to directly or indirectly connect those structures together. For example, and as shown in FIG. 5, the leg body 556 penetrates through at least one leaflet (here, first valve leaflet 104) and into, and optionally through, a wall 558 of the subvalvular portion 122 and/or the anchor portion 132 to connect the at least one leaflet to the subvalvular device 120.

The second leg end 554 thus may become located on an interior side of the wall 558, or may be at least partially embedded within the wall 558. (The “interior” side, as used herein, could be any side of any wall of the target structure-cardiac tissue and/or a portion of the subvalvular device 120.) Once it is within or “through” (on the interior side of) the wall 558, the second leg end 554 may splay-shift or pull in a predetermined direction—with respect to the first leg end 552 to resist egress from the target structure, which again could be cardiac tissue, at least a portion of the subvalvular device 120, or any other desired component of the apparatus 100 or the patient's heart. This splaying acts to spread the leg 550 in a “staple”-type manner and thus resist pullout when tensile force is applied to the fastener head 546. In many of the Figures, a plurality of legs 550 are provided to the fastener 542, to heighten the staple-like effect produced by the splaying of the second leg ends 554 apart from one another, and thus assist in maintaining the fastener anchor 544 in position in the target structure.

This splaying action of the second leg end 554 may be accomplished in any desired manner. For example, a tool could be provided to bend the leg body 556 or another structure of the fastener anchor 544 to carry out the splaying action. It is contemplated that the second leg end 554 may splay or shift with respect to the first leg end 552 by at least one of being made of a shape memory material and being elastically biased toward the splayed position. In the latter situation, the leg body 556 could be produced in a curved or pre-splayed configuration, elastically deformed (not plastically deformed) under pressure for insertion into the heart in a constrained condition, and then be released from the constraint (e.g., a catheter tube) to regain the curved, splayed configuration once placed into the desired relationship with the wall 558.

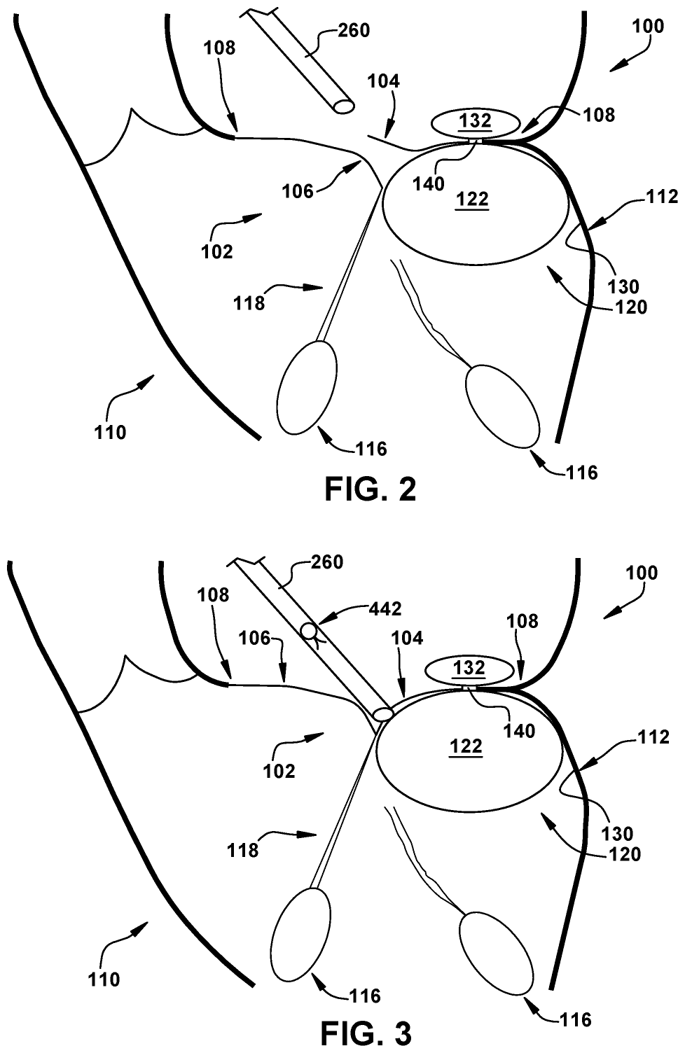

FIGS. 1-4 schematically depict a sequence of a method of at least partially repairing a regurgitant heart valve 102, having a flail subtype with the first valve leaflet 104 being separated from the chordae tendineae 118 and at least partially prolapsed. With reference to FIG. 1, a subvalvular device 120 is deployed with at least a portion of the subvalvular portion 122 adjacent a subvalvular cardiac wall 130 adjacent to the heart valve 102. The connector neck 140 is penetrating longitudinally through at least one of a base of the first valve leaflet 104 and an annulus 108 of the heart valve 102 at a manufactured puncture site. The subvalvular device 120 may reach the position shown in FIG. 1 in any desired manner, such as, for example, collapsing the subvalvular device 120 into a collapsed condition for insertion into the heart 110 via an elongated catheter (shown schematically at 260 in FIG. 2), in a transseptal approach. Once the subvalvular device 120 has traveled to a desired position adjacent the heart valve 192, the subvalvular device 120 may be expanded from the collapsed condition to an expanded condition. Further information regarding suitable schemes for placing the subvalvular device 120 into the position of any of the relevant Figures can be found in the disclosures of the '432 family, or can be provided by one of ordinary skill in the art for a particular use environment.

A fastener 442 is provided and inserted into the heart 110. For example, and as shown in the sequence of FIGS. 3-4, the fastener 442 may be collapsed into a collapsed condition for insertion into the heart 110 via an elongated catheter 260 (which could be the same as, or different than, the elongated catheter provided for insertion and deployment of the subvalvular device 120). Adjacent the subvalvular device 120, the fastener 442 can be expanded from the collapsed condition to an expanded condition.

Before, during, or after the expansion of the fastener 442 from the collapsed condition, the fastener 442 may be selectively connected, directly or indirectly, to the at least one subvalvular device 120. This connection, for example, may include selectively connecting the fastener 442 to a selected one of the subvalvular portion 122 and the anchor portion 132, in the depicted sequence, and optionally by way of the fastener anchor 544 being penetrated at least partially into at least a portion of the subvalvular device 120. For example, the fastener anchor 544 could engage a mesh forming at least a portion of the at least one subvalvular device 120. That is, the leg body 556 could extend through an aperture in the mesh, and the second leg end 554 could then splay outward to make the leg body 556 reach out too widely to be easily pulled back through the aperture.

It is contemplated that the fastener 442 could also or instead be fastened to at least one cardiac tissue. For example, and as particularly shown in FIG. 4, the fastener 442 is connected to at least one of the first and second valve leaflets 104 and 106 (here, to first valve leaflet 104). This fastening to cardiac tissue can include selectively engaging the fastener head 546 with the cardiac tissue. Again with reference to FIG. 4, the legs 550 of the fastener extend through the first valve leaflet 104, but the fastener head 546 is too large to fit through the punctured tissue behind the legs 550, so the fastener head 546 is considered to “engage” the first valve leaflet by pinning it to the subvalvular portion 122. In order to maintain the arrangement of FIG. 4, the leg body 556 is penetrated through the cardiac tissue (here, first valve leaflet 104) and a wall 558 of the subvalvular device 120 to connect the first valve leaflet 104 to the at least one subvalvular device 120—here, to the subvalvular portion 122. The second leg end(s) 554 is/are then splayed, on an interior side of the wall 558, with respect to the first leg end(s) 556 to resist egress of the fastener 442 from the at least one subvalvular device 120.

The first valve leaflet 104 can then be maintained in a predetermined position with respect to the at least one subvalvular device 120 via the fastener 442. In the use environment depicted schematically in FIGS. 1-4, the first valve leaflet 104 is “pinned” to the subvalvular device 120 by one or more fasteners 442 in lieu of the installation of replacement chordae tendineae to replace the ruptured or lost native chordae tendineae 118. The first valve leaflet 104 is thus supported by the subvalvular device 120 for desired coaptation with the second valve leaflet 106 due to usage of the apparatus 100.

Upon review of FIGS. 1-4 and the associated description, above, one of ordinary skill in the art will be readily able to extrapolate and develop various ways of installing one or more suitably configured subvalvular device(s) 120 and/or fastener(s) 442 to any desired cardiac tissue(s), to address or repair any of various undesirable heart 110 conditions. The sequence of installation of many of the following example embodiments of apparatuses 100 will not be described in detail, but can readily be determined by one of ordinary skill in the art, particularly in light of the method(s) which are described in some detail herein.

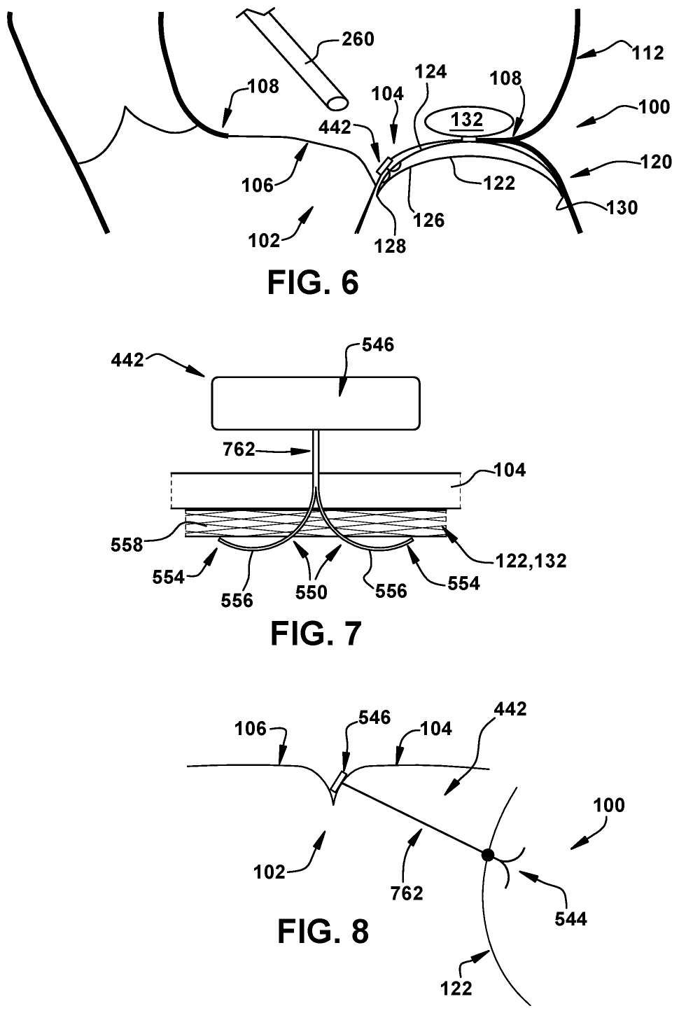

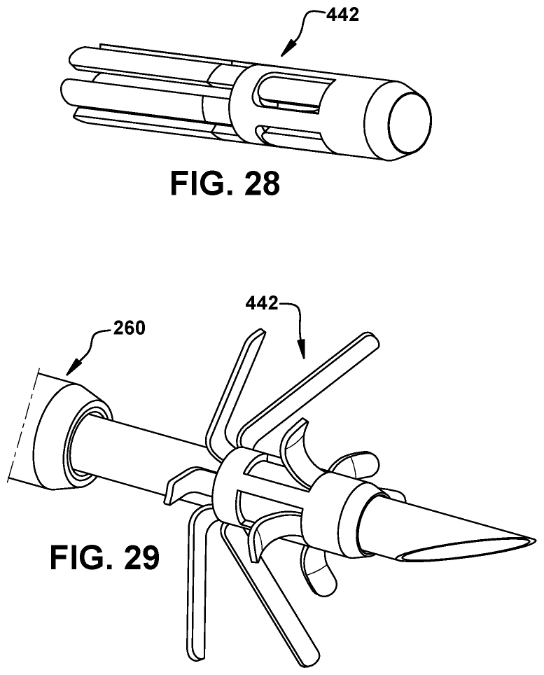

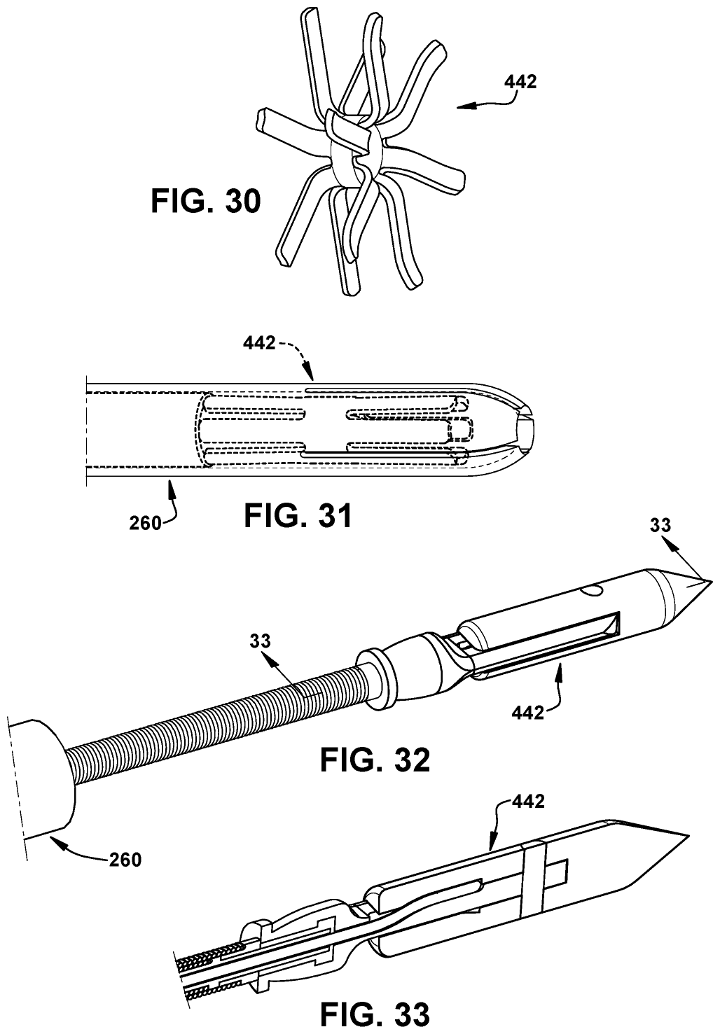

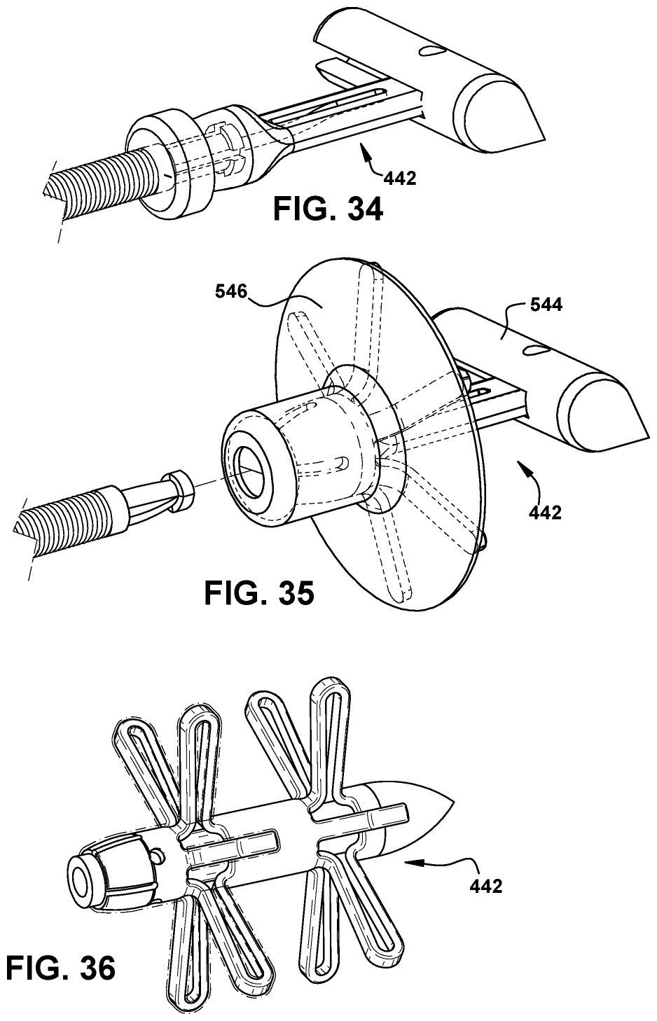

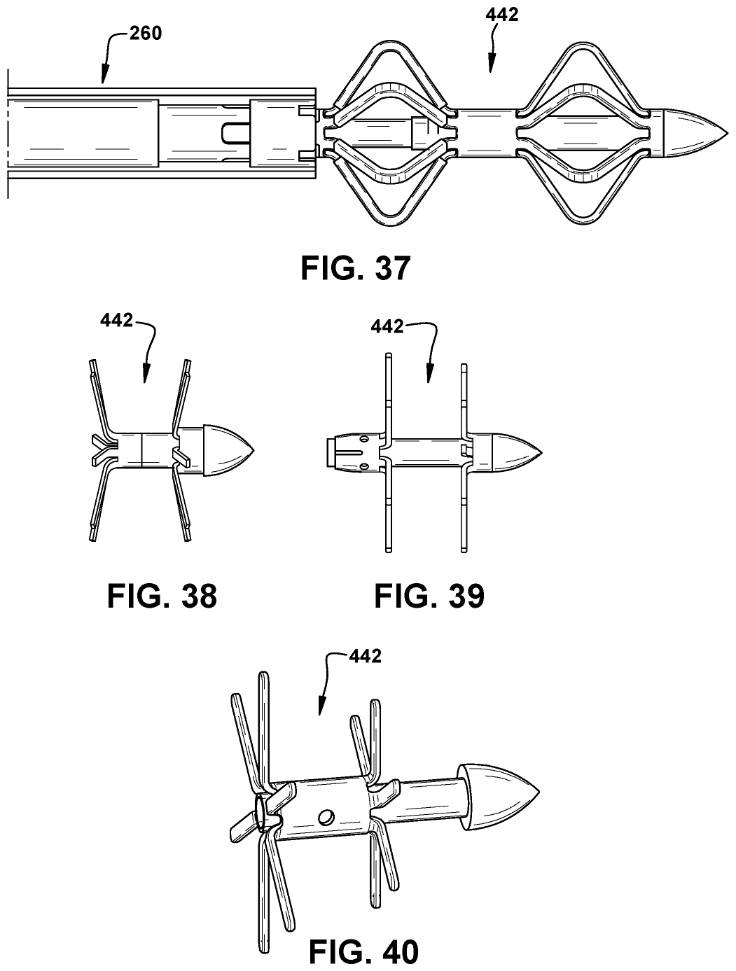

Certain structures of FIGS. 6-47 are similar to those of FIGS. 1-5. Therefore, structures of FIGS. 6-47 that are the same as or similar to those described with reference to FIGS. 1-5 will be numbered alike. Description of common elements and operation similar to those in the previously described embodiments will not be repeated with respect to the components, aspects, arrangements, and configurations described below, but should instead be considered to be incorporated below by reference as appropriate.

Turning now to FIG. 6, a “low profile” configuration of the subvalvular device 120 is shown. In the FIG. 6 configuration, the upper and lower subvalvular surfaces 124 and 126 are both convexly shaped. The subvalvular perimeter wall 128 is acting as a “lower rim” of a somewhat saucer-shaped subvalvular portion 122. The subvalvular perimeter wall 128 is, accordingly, located further longitudinally below the first valve leaflet 104 than are both of the upper and lower subvalvular surfaces 124 and 126.

Continuing to FIG. 7, an alternate configuration of a fastener 442 is shown schematically. In the fastener 442 of FIG. 6, the fastener anchor 544 is connected directly to the fastener head 546. In contrast, the fastener 442 of FIG. 7 has the fastener anchor 544 spaced apart from the fastener head 546 with a flexible tether 762 interposed mechanically therebetween. The tether 762 may be a wire, suture thread, fabric strip, or any other elongated, flexible material which is operable for secure attachment to the fastener anchor 544 and the fastener head 546, and which is suitable for withstanding tensile forces developed between the fastener anchor 544 and the fastener head 546, once the fastener 442 is installed into the heart 110.

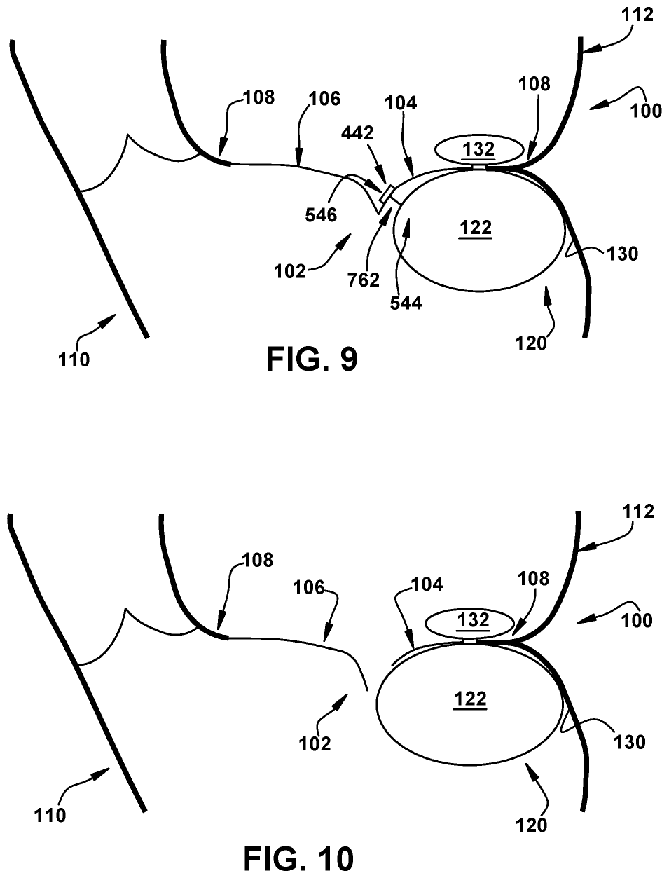

Attachment of the tether-type fastener 442 to the first valve leaflet 104 and the subvalvular portion 122 is shown schematically in FIG. 8, and in a larger heart 110 environment in the schematic view of FIG. 8. The flexible tether 762 may have a tether length which is predetermined (e.g., via preoperative imaging) before the fastener 442 is placed into maintaining contact with the cardiac tissue (e.g., at least one of the first and second valve leaflets 104 and 106) and/or with the at least one subvalvular device 120. Conversely, the tether length may be determined after at least a portion of the fastener 442 has been placed into maintaining contact with at least a chosen one of (1) the cardiac tissue (e.g., the first and second valve leaflets 104 and 106), and (2) the at least one subvalvular device 120 (e.g., the subvalvular portion 122). Length determination and/or adjustment of the tether 762 may be accomplished in any desired manner such as, but not limited to, pulling the fastener head 546 along the tether 762 at different distances from the fastener anchor 544 and then securing the fastener head 546 once a desired tether length is achieved. The tether 762 may serve as a pseudo-chordae and allow a predetermined amount of movement of the first valve leaflet 104 with respect to the subvalvular device 120 during the cardiac cycle, such as in the FIG. 9 arrangement.

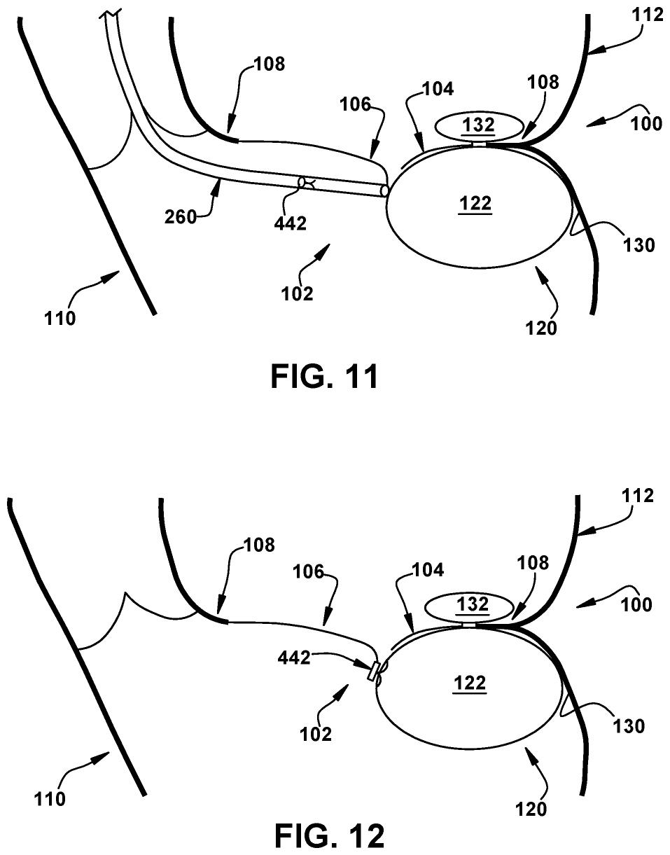

FIGS. 10-12 schematically depict an example use of the apparatus 100 to address mitral regurgitation where the first valve leaflet 104 is too short to repair by direct connection to the subvalvular device 120. In FIG. 11, the elongated catheter 260 is shown as being inserted into the heart 110 retro-aortically for installation of the fastener 442 from an underside of the second valve leaflet 106 and through that leaflet into the subvalvular portion 122.

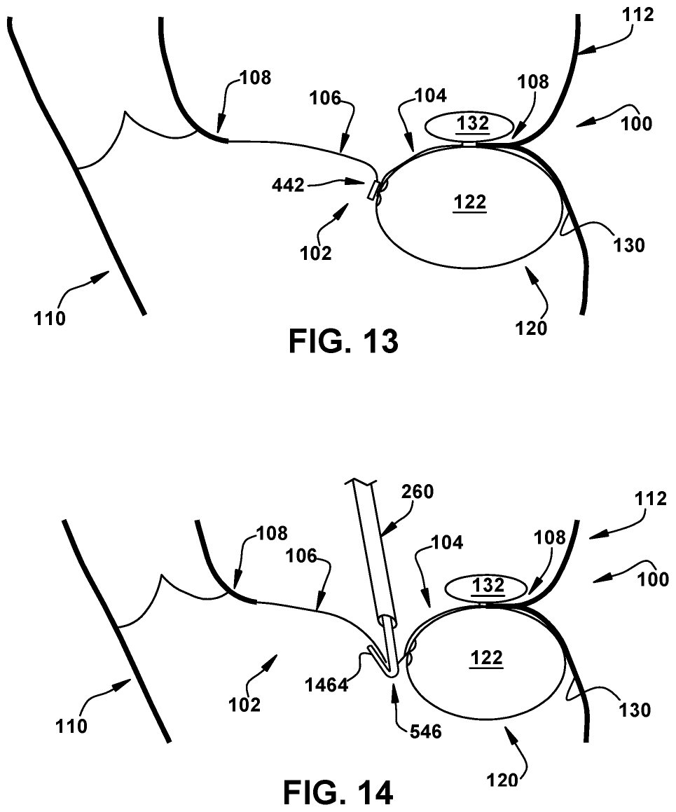

FIG. 13 schematically depicts an apparatus 100 having the fastener 442 penetrating concurrently through both of the first and second valve leaflets 104 and 106, to maintain both of the first and second valve leaflets 104 and 106 in predetermined positions with respect to each other and to the subvalvular device 120. The two-leaflet arrangement of FIG. 13 can be useful, for example, in a situation where the tethering of the first valve leaflet 104 is significant and the valve annulus 108 is sufficiently dilated that a traditional clip would be challenging or significantly distort the heart valve 102. Here, the subvalvular portion 122 is positioned under the first valve leaflet 104 to support or bolster it toward the second valve leaflet 106. The fastener 442 can then be advanced retro-aortically (similarly to the situation of FIGS. 10-12) to simultaneously maintain both of the first and second valve leaflets 104 and 106 in position with respect to the subvalvular device. The repair shown in FIG. 13 is therefore functionally equivalent to a known “edge-to-edge” repair technique.

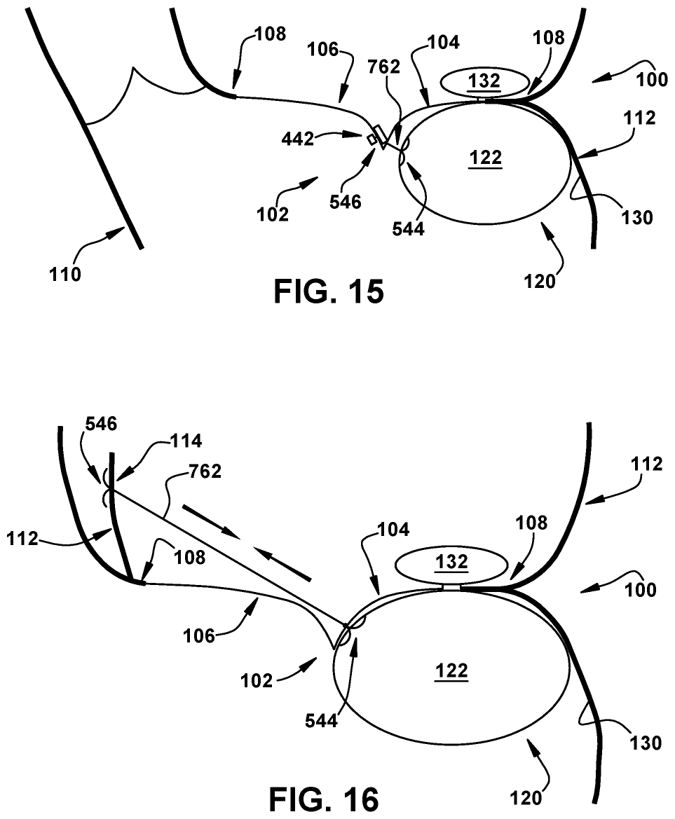

FIGS. 14-15 schematically depict an example sequence of installation of a fastener 442 having a tether 762 and a clip 1464 for engaging the second valve leaflet 106 for maintenance with respect to the subvalvular portion 122. As shown in FIG. 14, the fastener head 546 includes the clip 1464 for selectively grasping a free edge of at least one of the first and second valve leaflets 104 and 106 (here, the second valve leaflet 106). The clip 1464 may be manipulable by a wire (not shown) or other user-operable mechanism extending into the heart 110 via the elongate catheter 260 and eventually deployed after grasping the desired leaflet's free edge. The fastener anchor 544 could be deployed and secured into the subvalvular device 120 analogously to the previously described deployment, or in any other desired manner. In FIG. 15, the fastener 442 is shown as including a tether 762 for providing a limited amount of available movement space between the second leaflet 106 and subvalvular device 120.

With reference now to FIGS. 16-18, various installation configurations of a fastener 442 including a tether 762 (which could have a fixed length or be adjusted in situ, as previously described) are shown schematically. In FIGS. 16-18, the fastener 442 is used to exert tensile force across a heart chamber via engagement of the fastener head 546 with cardiac tissue spaced apart from the subvalvular device 120. For example, in FIG. 16, the fastener head 546 is selectively engaged with a myocardium 114 in a same heart chamber as the anchor portion 132 is located and the fastener anchor 544 is in selective attaching, optionally penetrative, contact with the subvalvular portion 122. To this end, the fastener head 546 may include one or more legs 550 splaying similarly to the leg(s) 550 of the fastener anchor 544 which engage with the subvalvular device 120. (Although it is contemplated that any desired structure including, but not limited to, one or more barbs, hooks, screws, adhesives, magnets, suture threads, staples, clips, pins, any other fastening means, or any combination thereof could be used to engage the fastener head 546 with the cardiac tissue.) Once arranged as in FIG. 16, the fastener 442 applies tensile force to urge the myocardium 114 toward the subvalvular portion 122 and responsively alter a shape of the valve annulus 108. As shown in FIG. 16, the fastener anchor 544 may penetrate through at least one of the first and second valve leaflets 104 and 106 (here, through first valve leaflet 104) and into selective penetrative contact with the subvalvular portion 122.

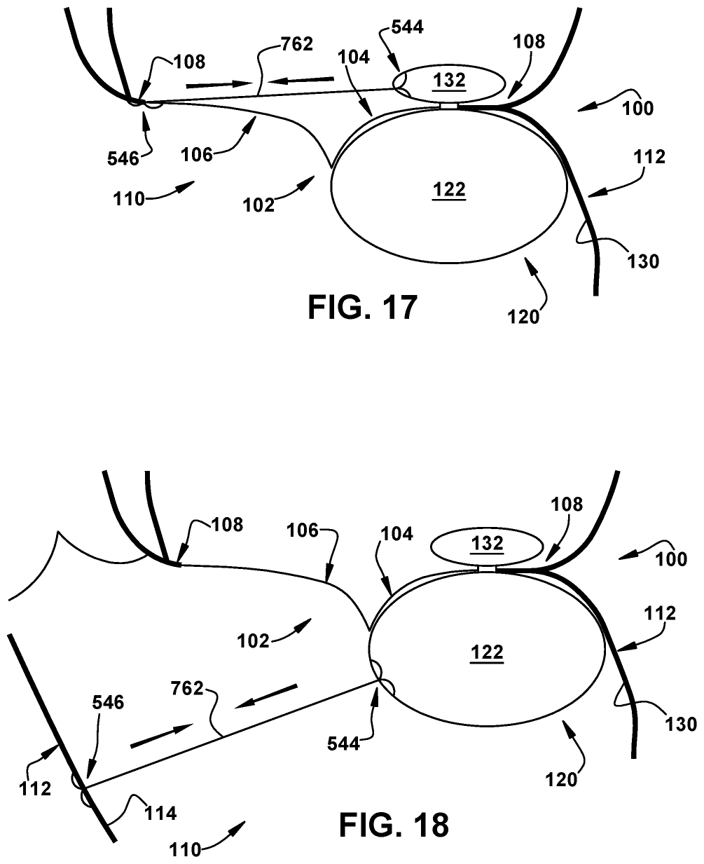

Turning to FIG. 17, the fastener head 546 is shown as being selectively engaged with the valve annulus 108 in a same heart chamber as the anchor portion 132 is located. Here, the fastener anchor 544 (and/or at least a portion of the tether 762) extends past at least one of the first and second valve leaflets 104 and 106 and into selective penetrative contact with the anchor portion 132. The fastener 442 applies tensile force to urge the valve annulus 108 toward the anchor portion 132 and responsively alter a shape of the valve annulus 108.

As shown in FIG. 18, the fastener head 546 may be selectively engaged with a myocardium 114 in a same heart chamber as the subvalvular portion 122 is located and the fastener anchor 544 extends into selective penetrative contact with the subvalvular portion 122. The fastener 442 (e.g., via the tether 762) applies tensile force to urge the myocardium 114 toward the subvalvular portion 122 and responsively alter a shape of the valve annulus. As with the configurations shown in FIGS. 16-17, the configuration of FIG. 18 could include at least a portion of the fastener 442 penetrating/passing through at least one of the first and second valve leaflets 104 and 106 in order to “pin” them into a desired spatial relationship with the remaining cardiac tissue and/or the subvalvular device 120.

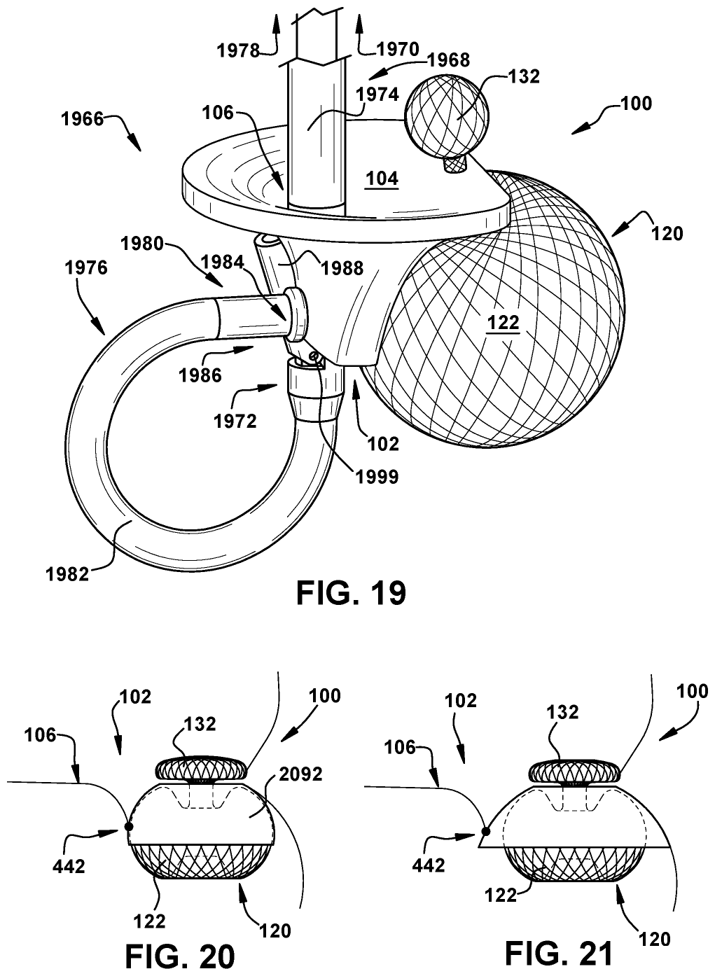

FIG. 19 depicts an apparatus 1966 for at least partially repairing a heart valve 102 of a patient. An elongate sheath 1968 has proximal and distal sheath ends 1970 and 1972, respectively, longitudinally spaced by a sheath body 1974 defining a sheath lumen. A fastener installation catheter 1976 has proximal and distal catheter ends 1978 and 1980, respectively, longitudinally spaced by a catheter body 1982 defining a catheter lumen. The catheter body 1982 is selectively insertable into the proximal sheath end 1970 and through the sheath lumen. While the distal sheath end 1972 is shown as being advanced up to, and even partially into, the orifice of the heart valve 102, it is contemplated that the distal sheath end 1972 may instead remain in the heart chamber spaced apart from the valve orifice while the catheter 1976 is advanced distally out of the distal sheath end 1972 through the valve orifice as described and shown.

The catheter 1976 includes a fastener release feature 1984 at the distal catheter end 1980. A fastener 442 may be carried within the catheter lumen and/or by the fastener release feature 1984, and one of ordinary skill in the art will be able to configure an apparatus 1966 appropriately for a particular use environment.

A grasping clip 1986 is connected to at least one of the distal sheath end 1972 and the catheter body 1982. The grasping clip 1986 is configured to selectively grasp at least one valve leaflet 104, 106 concurrently with the catheter 1976 extending through the valve orifice. The grasping clip 1986 includes a jaw member 1988 operable to pivot about a pivot point 1990 laterally with respect to the catheter body 1982. (“Laterally” here means toward and away from a central longitudinal axis defined through the catheter lumen.) The pivot point 1990 may be located at a distalmost end of the jaw member 1988, as shown in FIG. 19, for “backward” opening of the grasping clip 1986. Alternately, the pivot point 1990 may be located at a proximalmost end of the jaw member 1988, such that the grasping clip 1986 opens downward, toward the distal catheter end 1980.

While the at least one valve leaflet 104, 106 is being grasped by the grasping clip 1986, the catheter 1976 may be configured in a pigtail turn, as shown in FIG. 19, to place the fastener release feature 1984 into contact with the at least one valve leaflet 104, 106 from an underside thereof. Stated differently, when the grasping clip 1986 is connected to the distal sheath end 1972, the catheter body 1982 may be extendable from the distal sheath end 1972 to place the distal catheter end 1980 in substantially perpendicular orientation to the sheath body 1974, as shown in FIG. 19. The pigtail turn may be located distal to the grasping clip 1986, again as shown in FIG. 19.

Turning now to FIGS. 20-21, example arrangements of the subvalvular device 120 are shown. At least one of the subvalvular portion 122 and the anchor portion 132 includes a separately provided covering material 2092 attached to an outer surface thereof. The “outer surface” is used herein to indicate an area of the subvalvular portion 122 and/or anchor portion 132 which is exposed to ambient space. For example, the “outer surface” of the subvalvular device 122 can collectively comprise the upper and lower subvalvular surfaces 124 and 126 and the subvalvular perimeter wall 128, whereas the “outer surface” of the anchor portion 132 can collectively comprise the upper and lower anchor surfaces 134 and 136 and the anchor perimeter wall 138. This covering material 2092 is attached to at least one of an interior side (as a “lining”) and an exterior side (as a “cover”) of the outer surface of the subvalvular portion 122 and/or anchor portion 132. For example, in FIG. 20, the subvalvular portion 122 is shown having an exterior side of the outer surface being at least partially “covered” by the covering material 2092.

The covering material 2092 may be connected to the subvalvular device 122 at the connector neck 140 and selectively contact the outer surface of at least one of the subvalvular portion 122 and the anchor portion 132. For example, this is the situation shown in FIGS. 20-21. In FIG. 20, the covering material 2092 is substantially fitted to the outer surface of at least a portion of the subvalvular portion 122, and the fastener 442 penetrates through the covering material 2092 during selective connection to the subvalvular device 122.

In contrast, in the example arrangement of FIG. 21, at least a portion of the covering material 2092 is spaced apart from at least a portion of the exterior side of the outer surface of the subvalvular portion 122 with an intervening volume therebetween during at least a portion of a use cycle of the apparatus 100. In other words, the covering material 2092 may “billow” or “inflate” in a parachute-type manner during portions of the cardiac cycle, under hemodynamics from the blood flow within the heart 119. In this FIG. 21 arrangement, the fastener 442 may be connected to the subvalvular device 122 via direct connection to the covering material 2092. As a result, even without a tether 762 present, the parachute-type action of the covering material 2092 allows for some constrained amount movement of the attached valve leaflet 104, 106 with respect to the subvalvular portion 122 during the cardiac cycle.

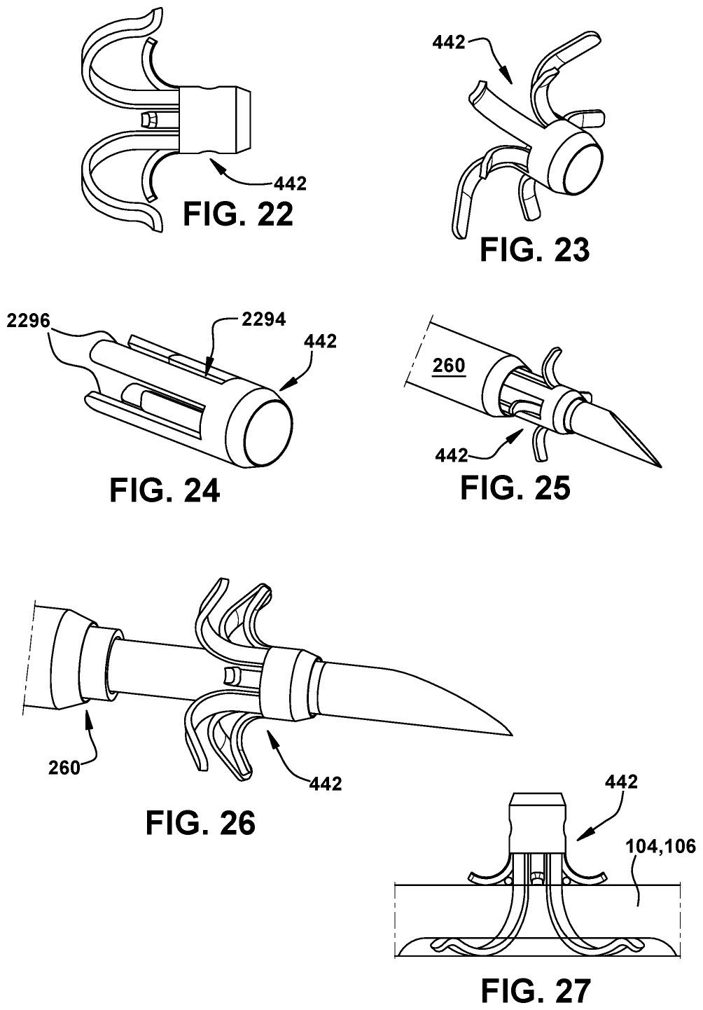

FIGS. 22-40 depict various example configurations, arrangements, and conditions of a number of example fasteners 442 which can be used with the apparatus 100. Several of the fasteners 442 shown in FIGS. 22-40 include a shape memory tube 2294 (e.g., a hypotube) with a plurality of slits 2294 therein defining at least one of the fastener anchor 544 and the fastener head 546. The slits 2294 may be, for example, laser-cut. Any desired arrangement of elongated (installation) catheter(s) 260 may be provided, and any desired deployment scheme can be employed. For example, the fastener 442 of FIGS. 22-27, including features of shape-memory material, can be carried on an introducer and deployed via pullback of a covering catheter 260. Furthermore, the fastener 442 could have a lumen (as can be seen in FIGS. 22-30) to allow for passing a needle, wire, or any other puncture instrument to help with puncturing through a first or second leaflet 104, 106 when delivering the fastener 442 into the subvalvular device 120. As another example, the fastener 442 of FIGS. 32-35 may include a pull-wire for deploying a pivoting toggle-type fastener anchor 544. Several of the fasteners 442 of FIGS. 22-40 include fastener heads 546 and fastener anchors 544 which substantially resemble one another in structure and can be used to “sandwich” together the structures (e.g., cardiac tissue and/or at least a portion of a subvalvular device 120) which are being attached together. One of ordinary skill in the art can readily provide one or more fasteners 442, each of which may be one of the designs depicted in the Figures or any other design, for a particular use environment of the present invention.

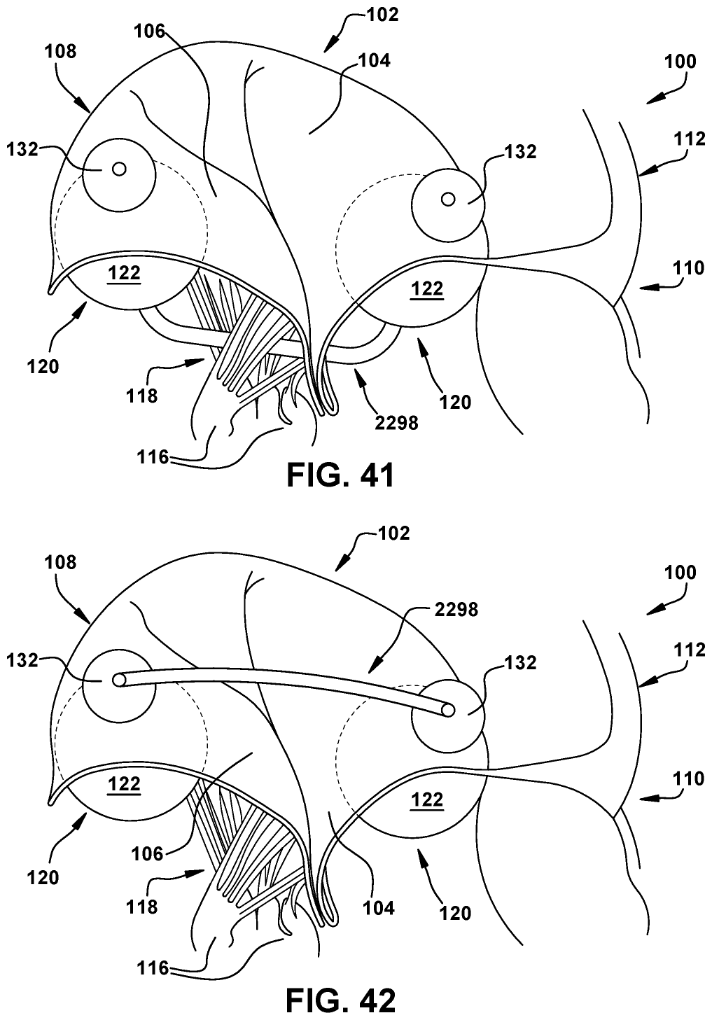

FIGS. 41-42 depict example configurations of the apparatus 100 which include a plurality of subvalvular devices 120 (here, first and second subvalvular devices 120), with each subvalvular device 120 being located on a manufactured puncture site on a different valve leaflet 104, 106 of the heart valve 102. A bridge member 2298 extends between, and mutually connects, the first and second subvalvular devices 120, responsively exerting tensile force therebetween to maintain each valve leaflet 104, 106 in a predetermined position with respect to the other valve leaflet 106, 104. The bridge member 2298 may be either substantially rigid or flexible.

In the FIG. 41 embodiment, the bridge member 2298 connects the subvalvular portions 122 of the first and second subvalvular devices 120 together and extends across an underside of the heart valve 102. In contrast, the FIG. 42 embodiment shows the bridge member 2298 connecting the anchor portions 132 of the first and second subvalvular devices 120 together and extends across an upper side of the heart valve 102.

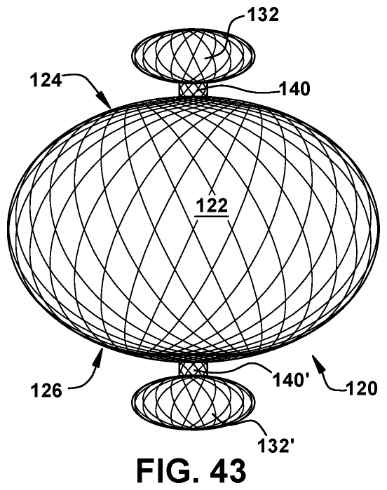

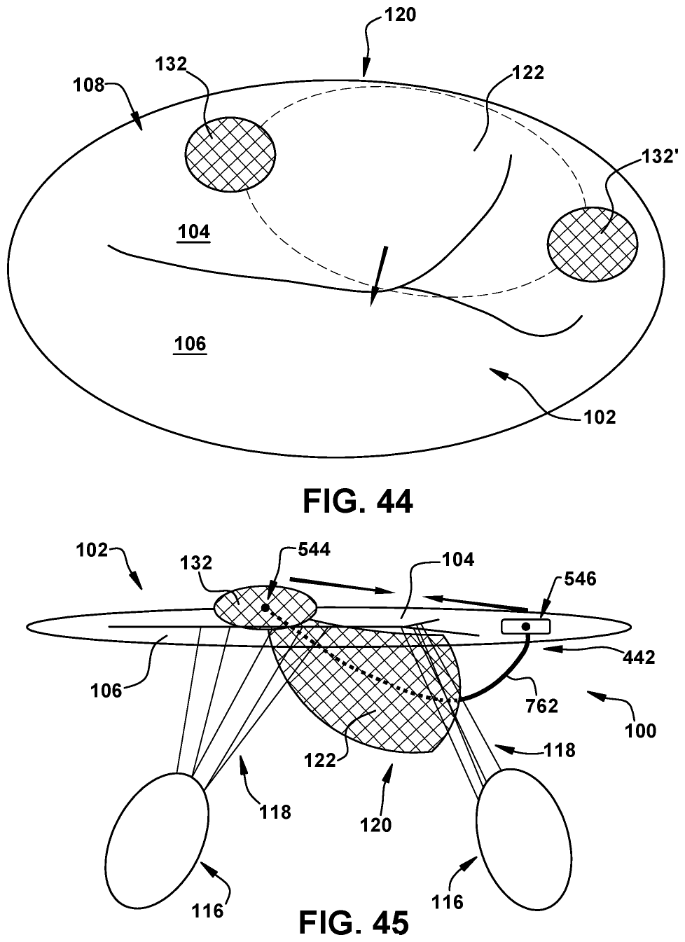

With reference to FIGS. 43-44, the anchor portion 132 may be a first anchor portion 132 and the subvalvular device 120 may include a second anchor portion 132′ adjacent to, and longitudinally spaced from, the lower subvalvular surface 126. When the subvalvular device 120 of FIGS. 43-44 is not adjacent the subvalvular cardiac wall 130 (i.e., is in the “rest position” shown in FIG. 43), the second anchor portion 132′ is spaced apart from the first anchor portion 132 with at least a portion of the subvalvular portion 122 interposed longitudinally therebetween. In contrast, when the subvalvular device 120 of FIG. 43 is shown in the installed configuration of FIG. 44, the first and second anchor portions 132 and 132′ are both located above the first valve leaflet 104, with connector necks 140, 140′ extending through at least one of the first valve leaflet 104 and the valve annulus 108 to hold the subvalvular portion 122 in place beneath the first valve leaflet 104 with two points of anchoring contact.

Finally, FIGS. 45-47 depict situations similar to those of FIGS. 16-18, in that the tether 762 of the fastener 442 spans across at least a portion of a heart chamber, for example, to help reshape the valve annulus 108 under tensile force between the subvalvular device 120 and the cardiac tissue with which the fastener head 546 is engaged. However, in the arrangements of FIGS. 45-47, the fastener 442 is selectively connected to a selected one of the subvalvular portion 122 and the anchor portion 132, and the flexible tether 762 extends through at least a portion of at least one of the anchor portion 132 and the subvalvular portion 122. By running the tether 762 through at least one of the anchor portion 132 and the subvalvular portion 122, the tether 762 can be used to position and support the thus-spanned anchor portion 132 and/or subvalvular portion 122 in a desired manner and orientation to appropriately support the valve leaflets, while providing an annuloplasty effect.

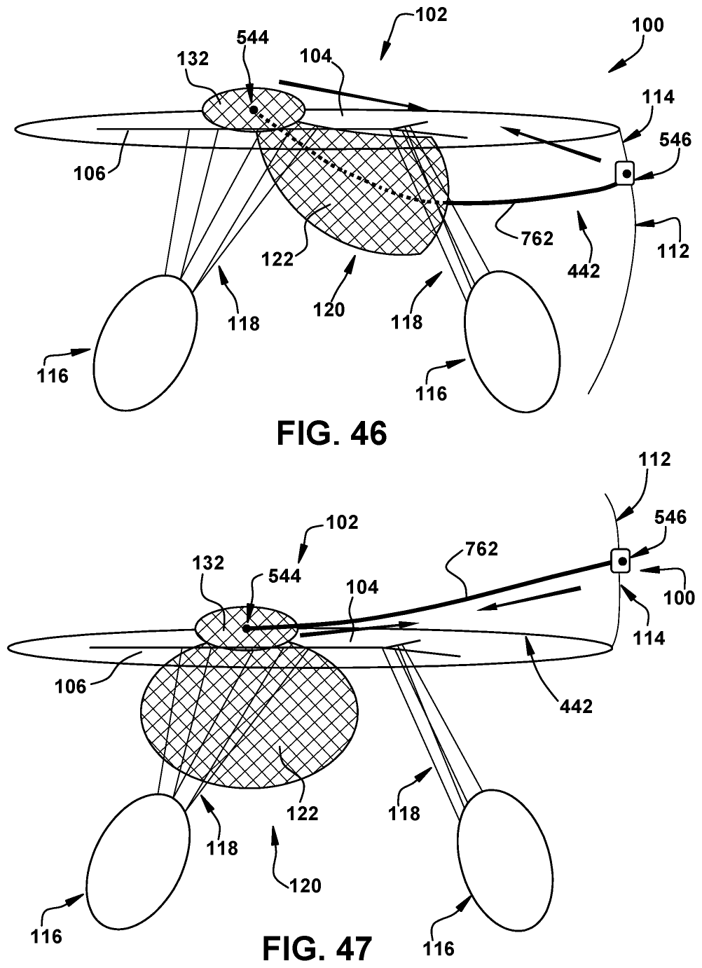

In FIG. 45, the fastener anchor 544 is engaged with the anchor portion 132, and the tether 762 extends through an interior volume of both the anchor portion 132 and the subvalvular portion 122 of the subvalvular device 120 to reach the fastener head 546, which is engaged with the valve annulus. FIG. 46 similarly depicts the fastener anchor 544 engaged with the anchor portion 132, and the tether 762 extends through an interior volume of both the anchor portion 132 and the subvalvular portion 122 of the subvalvular device 120 to reach the fastener head 546, which is engaged with the myocardium 114 of the heart wall 112 in the same heart chamber as the subvalvular portion 122. In contrast, FIG. 47 depicts the fastener anchor 544 engaged with the anchor portion 132, and the tether 762 extends from, and optionally through an interior volume of, the anchor portion 132 of the subvalvular device 120 to reach the fastener head 546, which is engaged with the myocardium 114 of the heart wall 112 in the same heart chamber as the anchor portion 132. Although not shown in FIGS. 45-47, it is contemplated that the tether 442, or any other structure of the fastener 442, could extend through, or otherwise be engaged with, the first and/or second valve leaflets 104 and 106 in the depicted arrangements, as desired. One of ordinary skill in the art can readily provide a suitable apparatus 100 having a tether 762 extending through, and between, any desired natural or artificial structures as desired for a particular use environment, upon review of the present disclosure.

In summary, a person having ordinary skill in the art will understand that an example aspect 1 includes a method of at least partially repairing a regurgitant heart valve, the heart valve including at least first and second valve leaflets, the method comprising:
providing at least one subvalvular device defining a longitudinal axis and including
a subvalvular portion located adjacent a subvalvular cardiac wall adjacent to the heart valve,
an anchor portion adjacent to, and longitudinally spaced from, the subvalvular portion, and
a connector neck interposed longitudinally between, and attached to both of, the subvalvular portion and the anchor portion;
deploying the subvalvular device with at least a portion of the subvalvular portion adjacent a subvalvular cardiac wall adjacent to the heart valve, the connector neck penetrating longitudinally through at least one of a base of the first valve leaflet and an annulus of the heart valve at a manufactured puncture site;
providing a fastener;
selectively connecting the fastener to the at least one subvalvular device,
connecting the fastener to at least one of the first and second valve leaflets; and
maintaining the at least one leaflet in a predetermined position with

respect to the at least one subvalvular device via the fastener.

Example aspect 2. The method of example aspect 1, wherein the

subvalvular portion includes a first-leaflet-adjacent upper subvalvular surface longitudinally spaced from an oppositely facing lower subvalvular surface, a subvalvular perimeter wall extending longitudinally between, and integrally and contiguously formed with both of, the upper and lower subvalvular surfaces;

wherein the anchor portion is adjacent to, and longitudinally spaced

from, the upper subvalvular surface, the anchor portion including a first-leaflet-adjacent lower anchor surface longitudinally spaced from an oppositely facing upper anchor surface and an anchor perimeter wall extending longitudinally between the upper and lower anchor surfaces, and

wherein the connector neck is interposed longitudinally between, and attached to both of, the upper subvalvular surface and the lower anchor surface; and

wherein deploying the subvalvular device with at least a portion of the subvalvular portion adjacent a subvalvular cardiac wall adjacent to the heart valve includes locating at least a portion of the subvalvular perimeter wall adjacent a subvalvular cardiac wall adjacent to the heart valve.

Example aspect 3. The method of example aspect 1, wherein selectively connecting the fastener to the at least one subvalvular device includes selectively connecting the fastener to a selected one of the subvalvular portion and the anchor portion.

Example aspect 4. The method of example aspect 1, including:
collapsing the subvalvular device into a collapsed condition for insertion into the heart via an elongated catheter; and
adjacent the heart valve, expanding the subvalvular device from the collapsed condition to an expanded condition.

Example aspect 5. The method of example aspect 1, including:
collapsing the fastener into a collapsed condition for insertion into the heart via an elongated catheter; and
adjacent the subvalvular device, expanding the fastener from the collapsed condition to an expanded condition.

Example aspect 6. The method of example aspect 1, wherein connecting the fastener to at least one of the first and second valve leaflets includes penetrating the fastener through at least one of the first and second valve leaflets.

Example aspect 7. The method of example aspect 1, wherein connecting the fastener to at least one of the first and second valve leaflets includes penetrating the fastener through both of the first and second valve leaflets to connect the first and second valve leaflets together.

Example aspect 8. The method of example aspect 1, wherein connecting the fastener to at least one of the first and second valve leaflets includes clipping at least a portion of the fastener to a free edge of at least one of the first and second valve leaflets.

Example aspect 9. The method of example aspect 1, wherein the fastener includes a fastener head and a fastener anchor,
wherein selectively connecting the fastener to a selected one of the subvalvular portion and the anchor portion includes placing the fastener anchor into selective penetrative contact with at least a portion of the subvalvular device, and
wherein connecting the fastener to at least one of the first and second valve leaflets includes selectively engaging the fastener head with the at least one of the first and second valve leaflets.

Example aspect 10. The method of example aspect 9, wherein the fastener anchor includes at least one elongate leg having first and second leg ends separated by a leg body, the first leg end being attached to the fastener head,
wherein maintaining the at least one leaflet in a predetermined position with respect to at least a portion of the subvalvular device via the fastener includes:
penetrating the leg body through the at least one of the first and second valve leaflets and a wall of the at least one subvalvular device to connect the at least one of the first and second valve leaflets to the at least one subvalvular device; and
splaying the second leg end, on an interior side of the wall, with respect to the first leg end to resist egress from the at least one subvalvular device.

Example aspect 11. The method of example aspect 10, wherein splaying the second leg end, on an interior side of the wall, with respect to the first leg end to resist egress from the at least one subvalvular device includes splaying the second leg end via at least one of the second leg end being made of a shape memory material and the second leg end being elastically biased toward the splayed position.

Example aspect 12. The method of example aspect 9, wherein placing

the fastener anchor into selective penetrative contact with at least a portion of the selected one of the subvalvular portion and the anchor portion includes engaging a mesh forming at least a portion of the at least one subvalvular device with the fastener anchor.

Example aspect 13. The method of example aspect 9, including spacing the fastener anchor apart from the fastener head with a flexible tether interposed mechanically therebetween.

Example aspect 14. The method of example aspect 13, including predetermining a tether length of the flexible tether before the fastener is placed into maintaining contact with the at least one of the first and second valve leaflets and the at least one subvalvular device.

Example aspect 15. The method of example aspect 13, including determining a tether length of the flexible tether after the fastener has been placed into maintaining contact with at least a chosen one of (1) the first and second valve leaflets, and (2) the at least one subvalvular device.

Example aspect 16. The method of example aspect 1, wherein connecting the fastener to at least one of the first and second valve leaflets includes:
providing an apparatus for installing a fastener associated with the subvalvular device in the heart valve of the patient, the apparatus including
an elongate sheath having proximal and distal sheath ends longitudinally spaced by a sheath body defining a sheath lumen,
a fastener installation catheter having proximal and distal catheter ends longitudinally spaced by a catheter body defining a catheter lumen, the catheter including a fastener release feature at the distal catheter end, and
a grasping clip connected to at least one of the distal sheath end and the catheter body;
selectively inserting the catheter body into the proximal sheath end and through the sheath lumen;
advancing the sheath lumen through a vasculature of the patient to place the distal sheath end adjacent the heart valve;
extending the distal catheter end from the distal sheath end;
extending the catheter body through the valve orifice to place the distal catheter end on an opposite side of the valve orifice from the proximal catheter end;
with the grasping clip, selectively grasping at least one valve leaflet concurrently with the catheter extending through the valve orifice;
with the catheter extending through the valve orifice and the at least one valve leaflet grasped with the grasping clip, routing at least the distal catheter end in a pigtail turn; and
placing the fastener release feature into contact with the at least one valve leaflet with the at least one valve leaflet interposed between the distal catheter end and the sheath body.

A person having ordinary skill in the art will understand that an example aspect 17 includes an apparatus for at least partially supporting a regurgitant heart valve, the heart valve including at least first and second valve leaflets, the apparatus comprising:
at least first and second subvalvular devices, each subvalvular device defining a longitudinal axis, each subvalvular device being associated with a respective first or second valve leaflet, and each subvalvular device including
a subvalvular portion including a leaflet-adjacent upper subvalvular surface longitudinally spaced from an oppositely facing lower subvalvular surface, a subvalvular perimeter wall extending longitudinally between, and integrally and contiguously formed with both of, the upper and lower subvalvular surfaces, at least a portion of the subvalvular perimeter wall contacting a subvalvular cardiac wall adjacent to the heart valve,
an anchor portion adjacent to, and longitudinally spaced from, the upper subvalvular surface, the anchor portion including a leaflet-adjacent lower anchor surface longitudinally spaced from an oppositely facing upper anchor surface and an anchor perimeter wall extending longitudinally between the upper and lower anchor surfaces, and
a connector neck interposed longitudinally between, and attached to both of, the upper subvalvular surface and the lower anchor surface, the connector neck penetrating longitudinally through at least one of a base of the respective leaflet and an annulus of the heart valve at a manufactured puncture site; and
a bridge member extending between, and mutually connecting, the first and second subvalvular devices, responsively exerting tensile force therebetween to maintain each leaflet in a predetermined position with respect to the other leaflet.

A person having ordinary skill in the art will understand that an example aspect 17 includes an apparatus for at least partially repairing a regurgitant heart valve, the heart valve including at least first and second valve leaflets carried in a valve annulus, and the heart valve being located in a heart having an outer wall including a myocardium, wherein the heart valve, first and second valve leaflets, valve annulus, and myocardium all comprise cardiac tissue, the apparatus comprising:
at least one subvalvular device defining a longitudinal axis and including
a subvalvular portion located adjacent a subvalvular cardiac wall adjacent to the heart valve,
an anchor portion adjacent to, and longitudinally spaced from, the subvalvular portion, and
a connector neck interposed longitudinally between, and attached to both of, the subvalvular portion and the anchor portion, the connector neck penetrating longitudinally through at least one of a base of the first valve leaflet and an annulus of the heart valve at a manufactured puncture site; and
a fastener selectively connected to the at least one subvalvular device, the fastener penetrating at least partially into a selected cardiac tissue to maintain the first and second valve leaflets in a predetermined position with respect to the valve annulus.

Example aspect 18. The apparatus of example aspect 17, wherein the subvalvular portion includes a first-leaflet-adjacent upper subvalvular surface longitudinally spaced from an oppositely facing lower subvalvular surface, a subvalvular perimeter wall extending longitudinally between, and integrally and contiguously formed with both of, the upper and lower subvalvular surfaces, at least a portion of the subvalvular perimeter wall being located adjacent a subvalvular cardiac wall adjacent to the heart valve;
wherein the anchor portion is adjacent to, and longitudinally spaced from, the upper subvalvular surface, the anchor portion including a first-leaflet-adjacent lower anchor surface longitudinally spaced from an oppositely facing upper anchor surface and an anchor perimeter wall extending longitudinally between the upper and lower anchor surfaces, and
wherein the connector neck is interposed longitudinally between, and attached to both of, the upper subvalvular surface and the lower anchor surface.

Example aspect 19. The apparatus of example aspect 17, wherein the fastener is selectively connected to a selected one of the subvalvular portion and the anchor portion.

Example aspect 20. The apparatus of example aspect 17, wherein the subvalvular portion and the anchor portion are both at least partially formed from at least one of braided mesh strands of a first configuration, braided mesh strands of a second configuration, a balloon, a plurality of longitudinally extending struts, and a plurality of laterally extending struts.

Example aspect 21. The apparatus of example aspect 17, wherein the subvalvular portion, anchor portion, and connector neck collectively enclose a single contiguous interior volume.

Example aspect 22. The apparatus of example aspect 18, wherein the upper and lower subvalvular surfaces are both convexly shaped, and the subvalvular perimeter wall is located further longitudinally below the first valve leaflet than are both of the upper and lower subvalvular surfaces.

Example aspect 23. The apparatus of example aspect 17, wherein the fastener penetrates through both of the first and second valve leaflets, to maintain both of the first and second valve leaflets in predetermined positions with respect to each other and to the valve annulus.

Example aspect 24. The apparatus of example aspect 18, wherein the anchor portion is a first anchor portion, and wherein the subvalvular device includes a second anchor portion adjacent to, and longitudinally spaced from, the upper subvalvular surface, the second anchor portion including a first-leaflet-adjacent lower second anchor surface longitudinally spaced from an oppositely facing upper second anchor surface and a second anchor perimeter wall extending longitudinally between the upper and lower second anchor surfaces.

Example aspect 25. The apparatus of example aspect 24, wherein, when the subvalvular device is not contacting the subvalvular cardiac wall, the second anchor portion is spaced apart from the first anchor portion with at least a portion of the subvalvular portion interposed longitudinally therebetween.

Example aspect 26. The apparatus of example aspect 17, wherein the fastener includes at least one of a suture thread, a pledget, a clip, a staple, and a leg.

Example aspect 27. The apparatus of example aspect 17, wherein the fastener includes a flexible covering over at least a portion of an outer fastener surface.

Example aspect 28. The apparatus of example aspect 17, wherein the fastener includes a fastener anchor for selective penetrative contact with the at least one subvalvular device, and a fastener head for selective engagement with at least a selected portion of the cardiac tissue.

Example aspect 29. The apparatus of example aspect 28, wherein the fastener includes a shape memory tube with a plurality of slits therein defining at least one of the fastener anchor and the fastener head.

Example aspect 30. The apparatus of example aspect 28, wherein the fastener head includes a clip for selectively grasping a free edge of at least one of the first and second valve leaflets.

Example aspect 31. The apparatus of example aspect 28, wherein the fastener anchor includes at least one elongate leg having first and second leg ends separated by a leg body, the first leg end being attached to the fastener head, the leg body penetrating through a wall of the at least one subvalvular device to connect at least the selected portion of the cardiac tissue to the at least one subvalvular device, the second leg end, on an interior side of the wall, splaying with respect to the first leg end to resist egress from the interior of the wall.

Example aspect 32. The apparatus of example aspect 31, wherein the second leg end splays with respect to the first leg end, within the interior side of the wall, via at least one of being made of a shape memory material and being elastically biased toward the splayed position.

Example aspect 33. The apparatus of example aspect 28, wherein the fastener anchor is connected directly to the fastener head.

Example aspect 34. The apparatus of example aspect 28, wherein the fastener anchor is spaced apart from the fastener head with a flexible tether interposed mechanically therebetween.

Example aspect 35. The apparatus of example aspect 34, wherein the flexible tether has a tether length which is predetermined before the fastener is placed into maintaining contact with the selected portion of the cardiac tissue and the at least one subvalvular device.

Example aspect 36. The apparatus of example aspect 34, wherein the flexible tether has a tether length which is determined after the fastener has been placed into maintaining contact with at least a chosen one of (1) the selected portion of the cardiac tissue, and (2) the at least one subvalvular device.

Example aspect 37. The apparatus of example aspect 34, wherein the fastener is selectively connected to a selected one of the subvalvular portion and the anchor portion, and the flexible tether extends through at least a portion of at least one of the anchor portion and the subvalvular portion.

Example aspect 38. The apparatus of example aspect 28, wherein the fastener head is selectively engaged with a myocardium in a same heart chamber as the anchor portion is located and the fastener anchor is in selective penetrative contact with the subvalvular portion, wherein the fastener applies tensile force to urge the myocardium toward the subvalvular portion and responsively alter a shape of the valve annulus.

Example aspect 39. The apparatus of example aspect 38, wherein the fastener tether penetrates through at least one of the first and second valve leaflets with the fastener anchor in penetrative contact with the subvalvular portion.

Example aspect 40. The apparatus of example aspect 28, wherein the fastener head is selectively engaged with the valve annulus in a same heart chamber as the anchor portion is located and the fastener anchor extends past at least one of the first and second valve leaflets into selective penetrative contact with the anchor portion, wherein the fastener applies tensile force to urge the valve annulus toward the anchor portion and responsively alter a shape of the valve annulus.

Example aspect 41. The apparatus of example aspect 28, wherein the fastener head is selectively engaged with a myocardium in a same heart chamber as the subvalvular portion is located and the fastener anchor extends into selective penetrative contact with the subvalvular portion, wherein the fastener applies tensile force to urge the myocardium toward the subvalvular portion and responsively alter a shape of the valve annulus.

Example aspect 42. The apparatus of example aspect 17, wherein at

least one of the subvalvular portion and the anchor portion includes a separately provided covering material attached to an outer surface thereof, on at least one of an interior side and an exterior side of the outer surface.

Example aspect 43. The apparatus of example aspect 42, wherein the covering material is connected to the subvalvular device at the connector neck and selectively contacts the outer surface of the at least one of the subvalvular portion and the anchor portion.

Example aspect 44. The apparatus of example aspect 42, wherein the covering material is substantially fitted to the outer surface of at least a portion of the subvalvular portion, and the fastener penetrates through the covering material during selective connection to the subvalvular device.

Example aspect 45. The apparatus of example aspect 42, wherein at least a portion of the covering material is spaced apart from at least a portion of the exterior side of the outer surface of the subvalvular portion with an intervening volume therebetween during at least a portion of a use cycle of the apparatus, and the fastener is connected to the subvalvular device via direct connection to the covering material.

A person having ordinary skill in the art will understand that an example aspect 46 includes an apparatus for at least partially repairing a heart valve of a patient, the heart valve including a valve annulus and at least two valve leaflets, collectively defining a valve orifice, the heart valve having an associated subvalvular device including a subvalvular portion, an anchor portion, and a fastener, the apparatus comprising:
an elongate sheath having proximal and distal sheath ends longitudinally spaced by a sheath body defining a sheath lumen;
a fastener installation catheter having proximal and distal catheter ends longitudinally spaced by a catheter body defining a catheter lumen, the catheter body being selectively insertable into the proximal sheath end and through the sheath lumen, the catheter including a fastener release feature at the distal catheter end; and
a grasping clip connected to at least one of the distal sheath end and the catheter body, the grasping clip being configured to selectively grasp at least one valve leaflet concurrently with the catheter extending through the valve orifice;
wherein, while the at least one valve leaflet is grasped by the grasping clip, the catheter is configured in a pigtail turn to place the fastener release feature into contact with the at least one valve leaflet.

Example aspect 47. The apparatus of example aspect 46, wherein the pigtail turn is located distal to the grasping clip.

Example aspect 48. The apparatus of example aspect 46, wherein the grasping clip includes a jaw member operable to pivot about a pivot point laterally with respect to the catheter body, the pivot point being located at a distalmost end of the jaw member.

Example aspect 49. The apparatus of example aspect 46, wherein the grasping clip includes a jaw member operable to pivot about a pivot point laterally with respect to the catheter body, the pivot point being located at a proximalmost end of the jaw member.

Example aspect 50. The apparatus of example aspect 46, wherein, when the grasping clip is connected to the distal sheath end, the catheter body is extendable from the distal sheath end to place the distal catheter end in substantially perpendicular orientation to the sheath body.

A person having ordinary skill in the art will understand that an example aspect 51 includes a method of at least partially repairing a regurgitant heart valve, the heart valve including a valve annulus and at least first and second valve leaflets, the method comprising:
providing a subvalvular device for installation in a heart valve of a patient, the subvalvular device including a subvalvular portion, an anchor portion, and a fastener;
providing an apparatus for installing a fastener associated with the subvalvular device in the heart valve of the patient, the apparatus including
an elongate sheath having proximal and distal sheath ends longitudinally spaced by a sheath body defining a sheath lumen,
a fastener installation catheter having proximal and distal catheter ends longitudinally spaced by a catheter body defining a catheter lumen, the catheter including a fastener release feature at the distal catheter end, and
a grasping clip connected to at least one of the distal sheath end and the catheter body;
selectively inserting the catheter body into the proximal sheath end and through the sheath lumen;
advancing the sheath through a vasculature of the patient to place the distal sheath end adjacent the heart valve;
extending the distal catheter end from the distal sheath end;
extending the catheter body through the valve orifice to place the distal catheter end on an opposite side of the valve orifice from the proximal catheter end;
with the grasping clip, selectively grasping at least one valve leaflet concurrently with the catheter extending through the valve orifice;
with the catheter extending through the valve orifice and the at least one valve leaflet grasped with the grasping clip, routing at least the distal catheter end in a pigtail turn; and
placing the fastener release feature into contact with the at least one valve leaflet with the at least one valve leaflet interposed between the distal catheter end and at least one of the catheter body and the sheath body.

Example aspect 52. The method of example aspect 51, wherein selectively grasping at least one valve leaflet concurrently with the catheter extending through the valve orifice includes:
pivoting a jaw member about a pivot point laterally with respect to the catheter body; and
maintaining at least a portion of the valve leaflet in compression between the jaw member and a chosen one of the catheter body and the sheath body.

Example aspect 53. The method of example aspect 51, wherein routing at least the distal catheter end in a pigtail turn includes:
when the grasping clip is connected to the distal sheath end, extending the catheter body from the distal sheath end to place the distal catheter end in substantially perpendicular orientation to the sheath body.

Example aspect 54. The method of example aspect 51, wherein routing at least the distal catheter end in a pigtail turn includes:
when the grasping clip is connected to the catheter body, turning the catheter body distal of the grasping clip to place the distal catheter end in substantially perpendicular orientation to the sheath body.

A person having ordinary skill in the art will understand that an example aspect 55 includes an apparatus for at least partially supporting a regurgitant heart valve, the heart valve including at least first and second valve leaflets, the apparatus comprising:
at least first and second subvalvular devices, each subvalvular device defining a longitudinal axis, each subvalvular device being associated with a respective first or second valve leaflet, and each subvalvular device including
a subvalvular portion including a leaflet-adjacent upper subvalvular surface longitudinally spaced from an oppositely facing lower subvalvular surface, a subvalvular perimeter wall extending longitudinally between, and integrally and contiguously formed with both of, the upper and lower subvalvular surfaces, at least a portion of the subvalvular perimeter wall contacting a subvalvular cardiac wall adjacent to the heart valve,
an anchor portion adjacent to, and longitudinally spaced from, the upper subvalvular surface, the anchor portion including a leaflet-adjacent lower anchor surface longitudinally spaced from an oppositely facing upper anchor surface and an anchor perimeter wall extending longitudinally between the upper and lower anchor surfaces, and
a connector neck interposed longitudinally between, and attached to both of, the upper subvalvular surface and the lower anchor surface, the connector neck penetrating longitudinally through at least one of a base of the respective leaflet and an annulus of the heart valve at a manufactured puncture site; and
a bridge member extending between, and mutually connecting, the first and second subvalvular devices, responsively exerting tensile force therebetween to maintain each leaflet in a predetermined position with respect to the other leaflet.

Example aspect 56. The apparatus of example aspect 55, wherein the subvalvular portion and the anchor portion are both at least partially formed from at least one of braided mesh strands of a first configuration, braided mesh strands of a second configuration, a balloon, a plurality of longitudinally extending struts, and a plurality of laterally extending struts.

Example aspect 57. The apparatus of example aspect 55, wherein the subvalvular portion, anchor portion, and connector neck collectively enclose a single contiguous interior volume.

Example aspect 58. The apparatus of example aspect 55, wherein the

upper and lower subvalvular surfaces are both convexly shaped, and the subvalvular perimeter wall is located further longitudinally below the respective leaflet than are both of the upper and lower subvalvular surfaces.

Example aspect 59. The apparatus of example aspect 58, wherein at

least one of the subvalvular portion and the anchor portion includes a separately provided covering material attached to a wall thereof, on at least one of an interior side and an exterior side of the wall.

Example aspect 60. The apparatus of example aspect 59, wherein the covering material is connected to the subvalvular device at the connector neck and selectively contacts the exterior side of the wall of the at least one of the subvalvular portion and the anchor portion.

Example aspect 61. The apparatus of example aspect 55, wherein the bridge member is substantially rigid.

Example aspect 62. The apparatus of example aspect 55, wherein the bridge member is flexible.

Example aspect 63. The apparatus of example aspect 55, wherein the bridge member connects the subvalvular portions of the first and second subvalvular devices together and extends across an underside of the heart valve.

Example aspect 64. The apparatus of example aspect 55, wherein the bridge member connects the anchor portions of the first and second subvalvular devices together and extends across an upper side of the heart valve.

While aspects of this disclosure have been particularly shown and described with reference to the example aspects above, it will be understood by those of ordinary skill in the art that various additional aspects may be contemplated. For example, the specific methods described above for using the apparatus are merely illustrative; one of ordinary skill in the art could readily determine any number of tools, sequences of steps, or other means/options for placing the above-described apparatus, or components thereof, into positions substantively similar to those shown and described herein. In an effort to maintain clarity in the Figures, certain ones of duplicative components shown have not been specifically numbered, but one of ordinary skill in the art will realize, based upon the components that were numbered, the element numbers which should be associated with the unnumbered components; no differentiation between similar components is intended or implied solely by the presence or absence of an element number in the Figures. Any of the described structures and components could be integrally formed as a single unitary or monolithic piece or made up of separate sub-components, with either of these formations involving any suitable stock or bespoke components and/or any suitable material or combinations of materials; however, the chosen material(s) should be biocompatible for many applications. Any of the described structures and components could be disposable or reusable as desired for a particular use environment. Any component could be provided with a user-perceptible marking to indicate a material, configuration, at least one dimension, or the like pertaining to that component, the user-perceptible marking potentially aiding a user in selecting one component from an array of similar components for a particular use environment. A “predetermined” status may be determined at any time before the structures being manipulated actually reach that status, the “predetermination” being made as late as immediately before the structure achieves the predetermined status. The term “substantially” is used herein to indicate a quality that is largely, but not necessarily wholly, that which is specified--a “substantial” quality admits of the potential for some relatively minor inclusion of a non-quality item. Though certain components described herein are shown as having specific geometric shapes, all structures of this disclosure may have any suitable shapes, sizes, configurations, relative relationships, cross-sectional areas, or any other physical characteristics as desirable for a particular application. Any structures or features described with reference to one aspect or configuration could be provided, singly or in combination with other structures or features, to any other aspect or configuration, as it would be impractical to describe each of the aspects and configurations discussed herein as having all of the options discussed with respect to all of the other aspects and configurations. A device or method incorporating any of these features should be understood to fall under the scope of this disclosure as determined based upon the claims below and any equivalents thereof.

Other aspects, objects, and advantages can be obtained from a study of the drawings, the disclosure, and the appended claims.

We claim:

1. An apparatus for at least partially repairing a regurgitant heart valve, the heart valve including at least first and second valve leaflets, the apparatus comprising:
at least one subvalvular device defining a longitudinal axis and including
a subvalvular portion located adjacent a subvalvular cardiac wall adjacent to the heart valve,
an anchor portion adjacent to, and longitudinally spaced from, the subvalvular portion, and
a connector neck interposed longitudinally between, and attached to both of, the subvalvular portion and the anchor portion, the connector neck penetrating longitudinally through at least one of a base of the first valve leaflet and an annulus of the heart valve at a manufactured puncture site; and
a fastener selectively connected to the at least one subvalvular device, the fastener penetrating through at least one of the first and second valve leaflets to maintain the at least one leaflet in a predetermined position with respect to the at least one subvalvular device.

2. The apparatus of claim 1, wherein the subvalvular portion includes a first-leaflet-adjacent upper subvalvular surface longitudinally spaced from an oppositely facing lower subvalvular surface, a subvalvular perimeter wall extending longitudinally between, and integrally and contiguously formed with both of, the upper and lower subvalvular surfaces, at least a portion of the subvalvular perimeter wall being located adjacent a subvalvular cardiac wall adjacent to the heart valve;
wherein the anchor portion is adjacent to, and longitudinally spaced from, the upper subvalvular surface, the anchor portion including a first-leaflet-adjacent lower anchor surface longitudinally spaced from an oppositely facing upper anchor surface and an anchor perimeter wall extending longitudinally between the upper and lower anchor surfaces, and
wherein the connector neck is interposed longitudinally between, and attached to both of, the upper subvalvular surface and the lower anchor surface.

3. The apparatus of claim 1, wherein the fastener is selectively connected to a selected one of the subvalvular portion and the anchor portion, the fastener penetrating through at least one of the first and second valve leaflets to maintain the at least one leaflet in a predetermined position with respect to the at least one subvalvular device.

4. The apparatus of claim 1, wherein the subvalvular portion and the anchor portion are both at least partially formed from at least one of braided mesh strands of a first configuration, braided mesh strands of a second configuration, a balloon, a plurality of longitudinally extending struts, and a plurality of laterally extending struts.

5. The apparatus of claim 1, wherein the subvalvular portion, anchor portion, and connector neck collectively enclose a single contiguous interior volume.

6. The apparatus of claim 2, wherein the upper and lower subvalvular surfaces are both convexly shaped, and the subvalvular perimeter wall is located further longitudinally below the first valve leaflet than are both of the upper and lower subvalvular surfaces.

7. The apparatus of claim 1, wherein the fastener penetrates through both of the first and second valve leaflets, to maintain both of the first and second valve leaflets in predetermined positions with respect to each other and to the subvalvular device.

8. The apparatus of claim 2, wherein the anchor portion is a first anchor portion, and wherein the subvalvular device includes a second anchor portion adjacent to, and longitudinally spaced from, the upper subvalvular surface, the second anchor portion including a first-leaflet-adjacent lower second anchor surface longitudinally spaced from an oppositely facing upper second anchor surface and a second anchor perimeter wall extending longitudinally between the upper and lower second anchor surfaces.

9. The apparatus of claim 8, wherein, when the subvalvular device is not adjacent the subvalvular cardiac wall, the second anchor portion is spaced apart from the first anchor portion with at least a portion of the subvalvular portion interposed longitudinally therebetween.

10. The apparatus of claim 1, wherein the fastener includes at least one of a suture thread, a pledget, a clip, a staple, and a leg.

11. The apparatus of claim 1, wherein the fastener includes a flexible covering over at least a portion of an outer fastener surface.

12. The apparatus of claim 1, wherein the fastener includes a fastener anchor for selective penetrative contact with the at least one subvalvular device, and a fastener head for selective engagement with the at least one of the first and second valve leaflets.

13. The apparatus of claim 12, wherein the fastener includes a shape memory tube with a plurality of slits therein defining at least one of the fastener anchor and the fastener head.

14. The apparatus of claim 12, wherein the fastener head includes a clip for selectively grasping a free edge of at least one of the first and second valve leaflets.

15. The apparatus of claim 12, wherein the fastener anchor includes at least one elongate leg having first and second leg ends separated by a leg body, the first leg end being attached to the fastener head, the leg body penetrating through the at least one of the first and second valve leaflets and a wall of the at least one subvalvular device to connect the at least one of the first and second valve leaflets to the at least one subvalvular device, the second leg end, on an interior side of the wall, splaying with respect to the first leg end to resist egress from the at least one subvalvular device.

16. The apparatus of claim 15, wherein the second leg end splays with respect to the first leg end, within the interior side of the wall, via at least one of being made of a shape memory material and being elastically biased toward the splayed position.

17. The apparatus of claim 12, wherein the fastener anchor is connected directly to the fastener head.

18. The apparatus of claim 12, wherein the fastener anchor is spaced apart from the fastener head with a flexible tether interposed mechanically therebetween.

19 The apparatus of claim 18, wherein the flexible tether has a tether length which is predetermined before the fastener is placed into maintaining contact with the at least one of the first and second valve leaflets and the at least one subvalvular device.

20. The apparatus of claim 18, wherein the flexible tether has a tether length which is determined after the fastener has been placed into maintaining contact with at least a chosen one of (1) the first and second valve leaflets, and (2) the at least one subvalvular device.

21. The apparatus of claim 18, wherein the fastener is selectively connected to a selected one of the subvalvular portion and the anchor portion, and the flexible tether extends through at least a portion of at least one of the anchor portion and the subvalvular portion.

22. The apparatus of claim 1, wherein at least one of the subvalvular portion and the anchor portion includes a separately provided covering material attached to an outer surface thereof, on at least one of an interior side and an exterior side of the outer surface.

23. The apparatus of claim 22, wherein the covering material is connected to the subvalvular device at the connector neck and selectively contacts the outer surface of the at least one of the subvalvular portion and the anchor portion.

24. The apparatus of claim 22, wherein the covering material is substantially fitted to the outer surface of at least a portion of the subvalvular portion, and the fastener penetrates through the covering material during selective connection to the subvalvular device.

25. The apparatus of claim 22, wherein at least a portion of the covering material is spaced apart from at least a portion of the exterior side of the outer surface of the subvalvular portion with an intervening volume therebetween during at least a portion of a use cycle of the apparatus, and the fastener is connected to the subvalvular device via direct connection to the covering material.
